(19) United States
(12) Patent Application Publication
(10) Pub. No.: US 2002/0071843 A1
(43) Pub. Date: Jun. 13, 2002

(54) Targeted therapeutic agents

(76) Inventors: King Chuen Li, Bethesda, MD (US); Mark David Bednarski, Los Altos, CA (US); Charles A. Wartchow, San Francisco, CA (US); John S. Pease, Los Altos, CA (US); Neal E. DeChene, Morgan Hill, CA (US); Julie Trulson, San Jose, CA (US); Zhi Min Shen, Palo Alto, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 09/976,254

(22) Filed: Oct. 11, 2001

Related U.S. Application Data
(60) Provisional application No. 60/239,684, filed on Oct. 11, 2000.

(51) Int. Cl. A61K 39/395

(52) U.S. Cl. 424/155.1

(57) ABSTRACT

Therapeutic and imaging agents which are comprised of a targeting entity, a therapeutic or treatment entity and a linking carrier are provided. The linking carrier imparts additional advantages to the therapeutic agents, which are not provided by conventional linking methods. Preferred agents of the present invention comprise a lipid construct, vesicle, liposome, or polymerized liposome. In some cases, the therapeutic or treatment entity is a radioisotope, chemotherapeutic agent, prodrug, toxin, or gene encoding a protein that exhibits cell toxicity. Preferably, the agent is further comprised of a stabilizing entity that imparts additional advantages to the therapeutic or imaging agent.

### FIELD OF THE INVENTION

This invention relates to therapeutic and imaging agents which are comprised of a targeting entity, a therapeutic or treatment entity and a linking carrier. Preferred agents of the present invention comprise a lipid construct, vesicle, liposome, or polymerized liposome. The therapeutic or treatment entity may be associated with the agent by covalent or non-covalent means. In some cases, the therapeutic or treatment entity is a radioisotope, chemotherapeutic agent, prodrug, toxin, or gene encoding a protein that exhibits cell toxicity. Preferably, the agent is further comprised of a stabilizing entity that imparts additional advantages to the therapeutic or imaging agent. The stabilizing entity may be associated with the agent by covalent or non-covalent means. Preferably, the stabilizing entity is dextran, which preferably forms a coating on the surface of the lipid construct, vesicle, liposome, or polymerized liposome. In preferred embodiments the linking carrier is a polymerized liposome. The linking carrier imparts additional advantages to the therapeutic agents, which are not provided by conventional linking methods.

### BACKGROUND OF THE INVENTION

Cancer remains one of the leading causes of death in the industrialized world. In the United States, cancer is the second most common cause of death after heart disease, accounting for approximately one-quarter of the deaths in 1997. Clearly, new and effective treatments for cancer will provide significant health benefits. Among the wide variety of treatments proposed for cancer, targeted therapeutic agents hold considerable promise. In principle, a patient could tolerate much higher doses of a cytotoxic agent if the cytotoxic agent is targeted specifically to cancerous tissue, as healthy tissue should be unaffected or affected to a much smaller extent than the pathological tissue.

Due to the high specificity of monoclonal antibodies, antibodies coupled to cytotoxic agents have been proposed for targeted cancer treatment therapies. Solid tumors, in particular, express certain antigens, on both the transformed cells comprising the tumor and the vasculature supplying the tumors, which are either unique to the tumor cells and vasculature, or overexpressed in tumor cells and vasculature in comparison to normal cells and vasculature. Thus, linking an antibody specific for a tumor antigen, or a tumor vasculature antigen, to a cytotoxic agent, should provide high specificity to the site of pathology. One group of such antigens is a family of proteins called cell adhesion molecules (CAMS), expressed by endothelial cells during a variety of physiological and disease processes. Reisfeld, “Monoclonal Antibodies in Cancer Immunotherapy,” Laboratory Immunology II, (1992) 12(2):201-216, and Archelos et al., “Inhibition of Experimental Autoimmune Encephalomyelitis by the Antibody to the Intercellular Adhesion Molecule ICAM-1,” Ann. of Neurology (1993) 34(2):145-154. Multiple endothelial ligands and receptors, including CAMs, are known to be upregulated during various pathologies, such as inflammation and neoplasia, and hence are attractive candidates for targeting strategies.

Other potential targets are integrins. Integrins are a group of cell surface glycoproteins that mediate cell adhesion and therefore are mediators of cell adhesion interactions that occur in various biological processes. Integrins are heterodimers composed of noncovalently linked α and β polypeptide subunits. Currently at least eleven different α subunits have been identified and at least six different β subunits have been identified. The various α subunits can combine with various β subunits to form distinct integrins. The integrin identified as αᵥβ₃ (also known as the vitronectin receptor) has been identified as an integrin that plays a role in various conditions or disease states including but not limited to tumor metastasis, solid tumor growth (neoplasia), osteoporosis, Paget's disease, humoral hypercalcemia of malignancy, angiogenesis, including tumor angiogenesis, antiangiogenesis, retinopathy, macular degeneration, arthritis, including rheumatoid arthritis, periodontal disease, psoriasis and smooth muscle cell migration (e.g., restenosis). Additionally, it has been found that such integrin inhibiting agents would be useful as antivirals, antifungals and antimicrobials. Thus, therapeutic agents that selectively inhibit or antagonize αᵥβ₃ would be beneficial for treating such conditions. It has been shown that the αᵥβ₃ integrin binds to a number of Arg-Gly-Asp (RGD) containing matrix macromolecules, such as fibrinogen (Bennett et al., Proc. Natl. Acad. Sci. USA, Vol. 80 (1983) 2417), fibronectin (Ginsberg et al., J. Clin. Invest., Vol. 71 (1983) 619-624), and von Willebrand factor (Ruggeri et al., Proc. Natl. Acad. Sci. USA, Vol. 79 (1982) 6038). Compounds containing the RGD sequence mimic extracellular matrix ligands so as to bind to cell surface receptors. However, it is also known that RGD peptides in general are non-selective for RGD dependent integrins. For example, most RGD peptides that bind to αᵥβ₃ also bind to αᵥβ₅, αᵥβ₁, and α_{IIb}β_{IIIa}. Antagonism of platelet α_{IIb}β_{IIIa} (also known as the fibrinogen receptor) is known to block platelet aggregation in humans.

A number of anti-integrin antibodies are known. Doerr, et al., J. Biol. Chem. 1996 271:2443 reported that a blocking antibody to αᵥβ₅ integrin in vitro inhibits the migration of MCF-7 human breast cancer cells in response to stimulation from IGF-1. Gui et al., British J. Surgery 1995 82:1192, report that antibodies against αᵥβ₁ and αᵥβ₅ inhibit in vitro chemoinvasion by human breast cancer carcinoma cell lines Hs578T and MDA-MB-231. Lehman et al., Cancer Research 1994 54:2102 show that a monoclonal antibody (69-6-5) reacts with several αᵥ integrins including αᵥβ₃ and inhibits colon carcinoma cell adhesion to a number of substrates, including vitronectin. Brooks et al., Science 1994 264:569 show that blockade of integrin activity with an anti-αᵥβ₃ monoclonal antibody inhibits tumor-induced angiogenesis of chick chorioallantoic membranes by human M21 melanoma fragments. Chuntharapai, et al., Exp. Cell. Res. 1993 205:345 disclose monoclonal antibodies 9G2.1.3 and IOC4.1.3 which recognize the αᵥβ₃ complex, the latter monoclonal antibody is said to bind weakly or not at all to tissues expressing αᵥβ₃ with the exception of osteoclasts and was suggested to be useful for in vivo therapy of bone disease. The former monoclonal antibody is suggested to have potential as a therapeutic agent in some cancers.

Ginsberg et al., U.S. Pat. No. 5,306,620 disclose antibodies that react with integrin so that the binding affinity of integrin for ligands is increased. As such these monoclonal antibodies are said to be useful for preventing metastasis by immobilizing melanoma tumors. Brown, U.S. Pat. No. 5,057,604 discloses the use of monoclonal antibodies to αᵥβ₃ integrins that inhibit RGD-mediated phagocytosis enhancement by binding to a receptor that recognizes RGD sequence containing proteins. Plow et al., U.S. Pat. No. 5,149,780 disclose a protein homologous to the RGD epitope of integrin β subunits and a monoclonal antibody that inhibits integrin-ligand binding by binding to the β₃ subunit. That action is said to be of use in therapies for adhesion-initiated human responses such as coagulation and some inflammatory responses.

Carron, U.S. Pat. No. 6,171,588, describe monoclonal antibodies which can be used in a method for blocking αᵥβ₃-mediated events such as cell adhesion, osteoclast-mediated bone resorption, restenosis, ocular neovascularization and growth of hemangiomas, as well as neoplastic cell or tumor growth and dissemination. Other uses described are antibody-mediated targeting and delivery of therapeutics for disrupting or killing αᵥβ₃ bearing neoplasms and tumor-related vascular beds. In addition, the inventive monoclonal antibodies can be used for visualization or imaging of αᵥβ₃-bearing neoplasms or tumor-related vascular beds by NMR or immunoscintigraphy.

Examples of the targeted therapeutic approach have been described in various patent publications and scientific articles. International Patent Application WO 93/17715 describes antibodies carrying diagnostic or therapeutic agents targeted to the vasculature of solid tumor masses through recognition of tumor vasculature-associated antigens. International Patent Application WO 96/01653 and U.S. Pat. No. 5,877,289 describe methods and compositions for in vivo coagulation of tumor vasculature through the site-specific delivery of a coagulant using an antibody, while International Patent Application WO 98/31394 describes use of Tissue Factor compositions for coagulation and tumor treatment. International Patent Application WO 93/18793 and U.S. Pat. Nos. 5,762,918 and 5,474,765 describe steroids linked to polyanionic polymers which bind to vascular endothelial cells. International Patent Application WO 91/07941 and U.S. Pat. No. 5,165,923 describe toxins, such as ricin A, bound to antibodies against tumor cells. U.S. Pat. Nos. 5,660,827, 5,776,427, 5,855,866, and 5,863,538 also disclose methods of treating tumor vasculature. International Patent Application WO 98/10795 and WO 99/13329 describe tumor homing molecules, which can be used to target drugs to tumors.

In Tabata, et al., Int. J. Cancer 1999 82:737-42, antibodies are used to deliver radioactive isotopes to proliferating blood vessels. Ruoslahti & Rajotte, Annu. Rev. Immunol. 2000 18:813-27; Ruoslahti, Adv. Cancer Res. 1999 76:1-20, review strategies for targeting therapeutic agents to angiogenic neovasculature, while Arap, et al., Science 1998 279:377-80 describe selection of peptides which target tumor blood vessels.

It should be noted that the typical arrangement used in such systems is to link the targeting entity to the therapeutic entity via a single bond or a relatively short chemical linker. Examples of such linkers include SMCC (succinimidyl 4-[N-maleimidomethyl]cyclohexane-1-carboxylate) or the linkers disclosed in U.S. Pat. No. 4,880,935, and oligopeptide spacers. Carbodiimides and N-hydroxysuccinimide reagents have been used to directly join therapeutic and targeting entities with the appropriate reactive chemical groups.

The use of cationic organic molecules to deliver heterologous genes in gene therapy procedures has been reported in the literature. Not all cationic compounds will complex with DNA and facilitate gene transfer. Currently, a primary strategy is routine screening of cationic molecules. The types of compounds which have been used in the past include cationic polymers such as polyethyleneamine, ethylene diamine cascade polymers, and polybrene. Proteins, such as polylysine with a net positive charge, have also been used. The largest group of compounds, cationic lipids; includes DOTMA, DOTAP, DMRIE, DC-chol, and DOSPA. All of these agents have proven effective but suffer from potential problems such as toxicity and expense in the production of the agents. Cationic liposomes are currently the most popular system for gene transfection studies. Cationic liposomes serve two functions: protect DNA from degradation and increase the amount of DNA entering the cell. While the mechanisms describing how cationic liposomes function have not been fully delineated, such liposomes have proven useful in both in vitro and in vivo studies. However, these liposomes suffer from several important limitations. Such limitations include low transfection efficiencies, expense in production of the lipids, poor colloidal stability when complexed to DNA, and toxicity.

Although conjugates of targeting entities with therapeutic entities via relatively small linkers have attracted much attention, far less attention has been focused on using large particles as linkers. Typically, the linker functions simply to connect the therapeutic and targeting entities, and consideration of linker properties generally focuses on avoiding interference with the entities linked, for example, avoiding a linkage point in the antigen binding site of an immunoglobulin.

Large particulate assemblies of biologically compatible materials, such as liposomes, have been used as carriers for administration of drugs and paramagnetic contrast agents. U.S. Pat. Nos. 5,077,057 and 5,277,914 teach preparation of liposome or lipidic particle suspensions having particles of a defined size, particularly lipids soluble in an aprotic solvent, for delivery of drugs having poor aqueous solubility. U.S. Pat. No. 4,544,545 teaches phospholipid liposomes having an outer layer including a modified, cholesterol derivative to render the liposome more specific for a preselected organ. U.S. Pat. No. 5,213,804 teaches liposome compositions containing an entrapped agent, such as a drug, which are composed of vesicle-forming lipids and 1 to 20 mole percent of a vesicle-forming lipid derivatized with hydrophilic biocompatible polymer and sized to control its biodistribution and recirculatory half life. U.S. Pat. No. 5,246,707 teaches phospholipid-coated microcrystalline particles of bioactive material to control the rate of release of entrapped water-soluble biomolecules, such as proteins and polypeptides. U.S. Pat. No. 5,158,760 teaches liposome encapsulated radioactive labeled proteins, such as hemoglobin.

U.S. Pat. Nos. 5,512,294 and 6,090,408, and 6,132,764 (the contents of which are hereby incorporated by reference herein) describe the use of polymerized liposomes for various biological applications. One listed embodiment is to targeted polymerized liposomes which may be linked to or may encapsulate a therapeutic compound, (e.g. proteins, hormones or drugs), for directed delivery of a treatment agent to specific biological locations for localized treatment. Other publications describing liposomal compositions include U.S. Pat. Nos. 5,663,387, 5,494,803, and 5,466,467. Liposomes containing polymerized lipids for non-covalent immobilization of proteins and enzymes are described in Storrs et al., “Paramagnetic Polymerized Liposomes: Synthesis, Characterization, and Applications for Magnetic Resonance Imaging,” J. Am. Chem. Soc. (1995) 117(28):7301-7306; and Storrs et al., “Paramagnetic Polymerized Liposomes as New Recirculating MR Contrast Agents,” JMRI (1995) 5(6):719-724. Wu et al., “Metal-Chelate-Dendrimer-Antibody Constructs for Use in Radioimmunotherapy and Imaging,” Bioorganic and Medicinal Chemistry Letters (1994) 4(3):449-454, is a publication directed to dendrimer-based compounds.

The need for recirculation of therapeutic agents in the body, that is avoidance of rapid endocytosis by the reticuloendothelial system and avoidance of rapid filtration by the kidney, to provide sufficient concentration at a targeted site to afford necessary therapeutic effect has been recognized. Experience with magnetic resonance contrast agents has provided useful information regarding circulation lifetimes. Small molecules, such as gadolinium diethylenetriaminepentaacetic acid, tend to have limited circulation times due to rapid renal excretion while most liposomes, having diameters greater than 800 nm, are quickly cleared by the reticuloendothelial system. Attempts to solve these problems have involved use of macromolecular materials, such as gadolinium diethylenetriaminepentaacetic acid-derived polysaccharides, polypeptides, and proteins. These agents have not achieved the versatility in chemical modification to provide for both long recirculation times and active targeting.

Stabilization

The association of liposomes with polymeric compounds in order to avoid rapid clearance in the liver, or for other stabilizing effects, has been described. For example, Dadey, U.S. Pat. No. 5,935,599 described polymer-associated liposomes containing a liposome, and a polymer having a plurality of anionic moieties in a salt form. The polymer may be synthetic or naturally-occurring. The polymer-associated liposomes remain in the vascular system for an extended period of time.

Polysaccharides are one class of polymeric stabilizer. Calvo Salve, et al., U.S. Pat. No. 5,843,509 describe the stabilization of colloidal systems through the formation of lipid-polysaccharide complexes and development of a procedure for the preparation of colloidal systems involving a combination of two ingredients: a water soluble and positively charged polysaccharide and a negatively-charged phospholipid. Stabilization occurs through the formation, at the interface, of an ionic complex: aminopolysaccharide-phospholipid. The polysaccharides utilized by Calvo Salve, et al., include chitin and chitosan.

Dextran is another polysaccharide whose stabilizing properties have been investigated. Cansell, et al., J. Biomed. Mater. Res. 1999, 44:140-48, report that dextran or functionalized dextran was hydrophobized with cholesterol, which anchors in the lipid bilayer of liposomes during liposome formation, resulting in a liposome coated with dextran. These liposomes interacted specifically with human endothelial cells in culture. In Letourneur, et al., J. Controlled Release 2000, 65:83-91, the antiproliferative functionalized dextran-coated liposomes were used as a targeting agent for vascular smooth muscle cells. Ullman, et al. Proc. Nat. Acad. Sci 91:5426-30 (1994) and Ullman, et al., Clin. Chem. 42:1518-26 (1996) describe the coating of polystyrene beads with dextran and the attachment of ligands, nucleic acids, and proteins to the dextran-polystyrene complexes.

Dextran has also been used to coat metal nanoparticles, and such nanoparticles have been used primarily as imaging agents. For example, Moore, et al., Radiology 2000, 214:568-74, report that in a rodent model, long-circulating dextran-coated iron oxide nanoparticles were taken up preferentially by tumor cells, but also were taken up by tumor-associated macrophages and, to a much lesser extent, endothelial cells in the area of angiogenesis. Groman, et al., U.S. Pat. No. 4,770,183, describe 10-5000 Å superparamagnetic metal oxide particles for use as imaging agents. The particles may be coated with dextran or other suitable polymer to optimize both the uptake of the particles and the residence time in the target organ. A dextran-coated iron oxide particle injected into a patient's bloodstream, for example, localizes in the liver. Groman, et al., also report that dextran-coated particles can be preferentially absorbed by healthy cells, with less uptake into cancerous cells.

Imaging

Magnetic resonance imaging (MRI) is an imaging technique which, unlike X-rays, does not involve ionizing radiation. MRI may be used for producing cross-sectional images of the body in a variety of scanning planes such as, for example, axial, coronal, sagittal or orthogonal. MRI employs a magnetic field, radio-frequency energy and magnetic field gradients to make images of the body. The contrast or signal intensity differences between tissues mainly reflect the T1 (longitudinal) and T2 (transverse) relaxation values and the proton density in the tissues. To change the signal intensity in a region of a patient by the use of a contrast medium, several possible approaches are available. For example, a contrast medium may be designed to change either the T1, the T2 or the proton density.

Generally speaking, MRI requires the use of contrast agents. If MRI is performed without employing a contrast agent, differentiation of the tissue of interest from the surrounding tissues in the resulting image may be difficult. In the past, attention has focused primarily on paramagnetic contrast agents for MRI. Paramagnetic contrast agents involve materials which contain unpaired electrons. The unpaired electrons act as small magnets within the main magnetic field to increase the rate of longitudinal (T1) and transverse (T2) relaxation. Paramagnetic contrast agents typically comprise metal ions, for example, transition metal ions, which provide a source of unpaired electrons. However, these metal ions are also generally highly toxic. For example, ferrites often cause symptoms of nausea after oral administration, as well as flatulence and a transient rise in serum iron. The gadolinium ion, which is complexed in Gd-DTPA, is highly toxic in free form. The various environments of the gastrointestinal tract, including increased acidity (lower pH) in the stomach and increased alkalinity (higher pH) in the intestines, may increase the likelihood of decoupling and separation of the free ion from the complex. In an effort to decrease toxicity, the metal ions are typically chelated with ligands.

Ultrasound is another valuable diagnostic imaging technique for studying various areas of the body, including, for example, the vasculature, such as tissue microvasculature. Ultrasound provides certain advantages over other diagnostic techniques. For example, diagnostic techniques involving nuclear medicine and X-rays generally involve exposure of the patient to ionizing electron radiation. Such radiation can cause damage to subcellular material, including deoxyribonucleic acid (DNA), ribonucleic acid (RNA) and proteins. Ultrasound does not involve such potentially damaging radiation. In addition, ultrasound is inexpensive relative to other diagnostic techniques, including CT and MRI, which require elaborate and expensive equipment.

Ultrasound involves the exposure of a patient to sound waves. Generally, the sound waves dissipate due to absorption by body tissue, penetrate through the tissue or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including the constituents and the density of the particular tissue being observed. Ultrasound involves the detection of the differentially reflected waves, generally with a transducer that can detect sound waves having a frequency of one to ten megahertz (MHz). The detected waves can be integrated into an image which is quantitated and the quantitated waves converted into an image of the tissue being studied.

As with the diagnostic techniques discussed above, ultrasound also generally involves the use of contrast agents. Exemplary contrast agents include, for example, suspensions of solid particles, emulsified liquid droplets, and gas-filled bubbles (see, e.g., Hilmann et al., U.S. Pat. No. 4,466,442, and published International Patent Applications WO 92/17212 and WO 92/21382). Widder et al., published application EP-A-0 324 938, disclose stabilized microbubble-type ultrasonic imaging agents produced from heat-denaturable biocompatible protein, for example, albumin, hemoglobin, and collagen.

The reflection of sound from a liquid-gas interface is extremely efficient. Accordingly, liposomes or vesicles, including gas-filled bubbles, are useful as contrast agents. As discussed more fully hereinafter, the effectiveness of liposomes as contrast agents depends upon various factors, including, for example, the size and/or elasticity of the bubble.

Many of the liposomes disclosed in the prior art have undesirably poor stability. Thus, the prior art liposomes are more likely to rupture in vivo resulting, for example, in the untimely release of any therapeutic and/or diagnostic agent contained therein. Various studies have been conducted in an attempt to improve liposome stability. Such studies have included, for example, the preparation of liposomes in which the membranes or walls thereof comprise proteins, such as albumin, or materials which are apparently strengthened via crosslinking. See, e.g., Klaveness et al., WO 92/17212, in which there are disclosed liposomes which comprise proteins crosslinked with biodegradable crosslinking agents. A presentation was made by Moseley et al., at a 1991 Napa, Calif. meeting of the Society for Magnetic Resonance in Medicine, which is summarized in an abstract entitled “Microbubbles: A Novel MR Susceptibility Contrast Agent.” The microbubbles described by Moseley et al. comprise air coated with a shell of human albumin. Alternatively, membranes can comprise compounds which are not proteins but which are crosslinked with biocompatible compounds. See, e.g., Klaveness et al., WO 92/17436, WO 93/17718 and WO 92/21382.

### SUMMARY OF THE INVENTION

This invention relates to therapeutic and imaging agents which are comprised of a targeting entity, a therapeutic or treatment entity and a linking carrier. Preferred agents of the present invention are comprised of a lipid construct, vesicle, liposome, or polymerized liposome. The therapeutic or treatment entity may be associated with the linking carrier by covalent or non-covalent means. In some cases, the therapeutic or treatment entity is a radioisotope, chemotherapeutic agent, prodrug, or toxin. In the most preferred embodiments, the linking carrier is a polymerized liposome. The linking carrier imparts additional advantages to the therapeutic agents, which are not provided by conventional linking methods.

The present invention is also directed toward vascular-targeted imaging agents comprised of a targeting entity, an imaging entity, and optionally, a linking carrier. The present invention is further directed toward diagnostic agents comprised of a targeting entity, a detection entity, and optionally, a linking carrier.

The present invention is also directed toward methods for preparing the aforementioned therapeutic and imaging agents.

The present invention is also directed toward therapeutic compositions comprising the therapeutic agents of the present invention.

The present invention is also directed toward methods of treatment utilizing the therapeutic agents of the present invention.

The present invention is also directed toward compositions for imaging comprising imaging agents of the present invention.

The present invention is also directed toward methods for utilizing the imaging agents of the present invention, including a method for diagnosing cancer.

The present invention is also directed toward methods and reagents for use in diagnostic assays.

### BRIEF DESCRIPTION OF THE FIGURES

FIG. 1 schematically shows the interaction of a vascular-targeted therapeutic agent with its target according to this invention;

FIGS. 2, 3, 4, and 29 schematically show polymerizable lipid molecules according to one embodiment of this invention;

FIG. 4 shows the synthesis of a metal chelated lipid according to one embodiment of this invention;

FIGS. 5 and 6 show formation of polymerized liposomes from the metal chelated lipid shown in FIG. 4 with filler lipids DAPC, DAPE or PDA according to one embodiment of this invention;

FIG. 7 shows the synthesis of biotinylated chelated lipids according to one embodiment of this invention;

FIGS. 8 and 9 show formation of biotinylated polymerized liposomes using PDA and DAPC or DAPE;

FIG. 10 shows formation of polymerized liposomes having positively charged functional groups;

FIG. 11 shows formation of polymerized liposomes having negatively charged functional groups;

FIG. 12 shows formation of polymerized liposomes having zwitterionic functional groups;

FIG. 13 shows formation of polymerized liposomes having lactose targeting groups;

FIG. 14 schematically shows formation of polymerized liposomes having antibodies attached where 71 is a liposome with a biotin surface, 72 is a biotin binding protein, and 70 and 74 comprise a biotinylated antibody;

FIGS. 15 and 16 show formation of liposomes that can be used for direct attachment of oxidized antibodies by an amine via reductive amination and hydrazone formation via alkyl hydrazine;

FIG. 17 is a schematic showing of an antibody-conjugated polymerized liposome as prepared in Example 9;

FIG. 18 is a photograph in color of gel electrophoresis using anti-avidin alkaline phosphatase as described in Example 10;

FIG. 19 is a photograph in color of gel electrophoresis using anti-IgG alkaline phosphatase as described in Example 10;

FIG. 20 is a fluorescence micrograph in color showing cell binding of fluorescent antibody-conjugated polymerized liposomes as described in Example 11;

FIG. 21 shows schematically the cell binding shown in FIG. 20;

FIG. 22 is a fluorescence micrograph in color of mouse cerebellum showing anti-ICAM-1 antibody-conjugated polymerized liposomes bound to capillaries as described in Example 12;

FIG. 23 is a magnetic resonance image of a brain slice of an experimental autoimmune encephalitis mouse without injection of polymerized liposomes as described in Example 13;

FIG. 24 is a magnetic resonance image of a brain slice of an experimental autoimmune encephalitis mouse injected with anti-ICAM-1 antibody-conjugated polymerized liposomes as described in Example 13;

FIG. 25 is a magnetic resonance image of a brain slice of a healthy mouse injected with anti-ICAM-1 antibody-conjugated polymerized liposomes as described in Example 13;

FIG. 26 is a bar chart showing magnetic resonance image intensity measurements as described in Example 13;

FIG. 27A shows MR images of V2 carcinoma in the thigh muscle of a rabbit and subcutaneously prior to (A), and at 24 hours post (B), anti -αᵥβ₃-labeled AbPV injection, while FIG. 27B shows MR images of isotype matched controls for FIG. 27A, as described in Example 23; and

FIG. 28A shows imaging of the Vx2 carcinoma with CPV-¹¹¹In conjugates in a rabbit model with non-targeting CPV-¹¹¹In.

FIG. 28B shows imaging of the Vx2 carcinoma with αᵥβ₃ integrin-targeted LM609-CPV-¹¹¹In, and reveals accumulation of the LM609-CPV-¹¹¹In complex in the tumor (lower left).

FIG. 29 shows structures for the triacetic acid chelator lipid [PDA-PEG₃]₂DTTA 5 and BisT-PC 6 (1,2-bis(10, 12 tricosadiynoyl)-sn-glycero-3-phosphocholine).

FIG. 30 shows radiometric αᵥβ₃ integrin binding assay for Vitaxin-CPV-⁹⁰Y complexes at yttrium-90 (⁹⁰Y) loadings of 0.16, 0.80, and 4 mCi of yttrium-90 per mg of Vitaxin-CPV conjugate. 96-well plates coated with human αᵥβ₃ integrin and blocked with 3% BSA were incubated with Vitaxin-CPV-⁹⁰Y or CPV-⁹⁰Y complexes for 1 h. The plates were washed and the yttrium-90 emission was determined with a scintillation plate reader.

FIG. 31 shows the effect of vesicle composition on the serum stability for a Vitaxin-CPV-⁹⁰Y conjugate containing chelator 5 and BisT-PC lipid 6 (5/95 molar ratio) and a Vitaxin-liposome-⁹⁰Y complex (Vitaxin-CL-⁹⁰Y) containing egg PC, cholesterol, and chelator 5 in molar ratios of 67/28/5 in rabbit serum at 37° C.

FIG. 32 shows the effect of yttrium-90 on the immunoreactivity of the Vitaxin-CPV complex relative to controls without yttrium and in the presence of 50 μM yttrium-89. Yttrium-90 loadings are expressed as mCi yttrium-90 per mg of vesicle. After labeling the vesicles, the complexes were stored at 4° C. for 60 days and assayed for binding to the αᵥβ₃ integrin by ELISA.

### DETAILED DESCRIPTION OF THE PREFERRED EMBODIMENTS

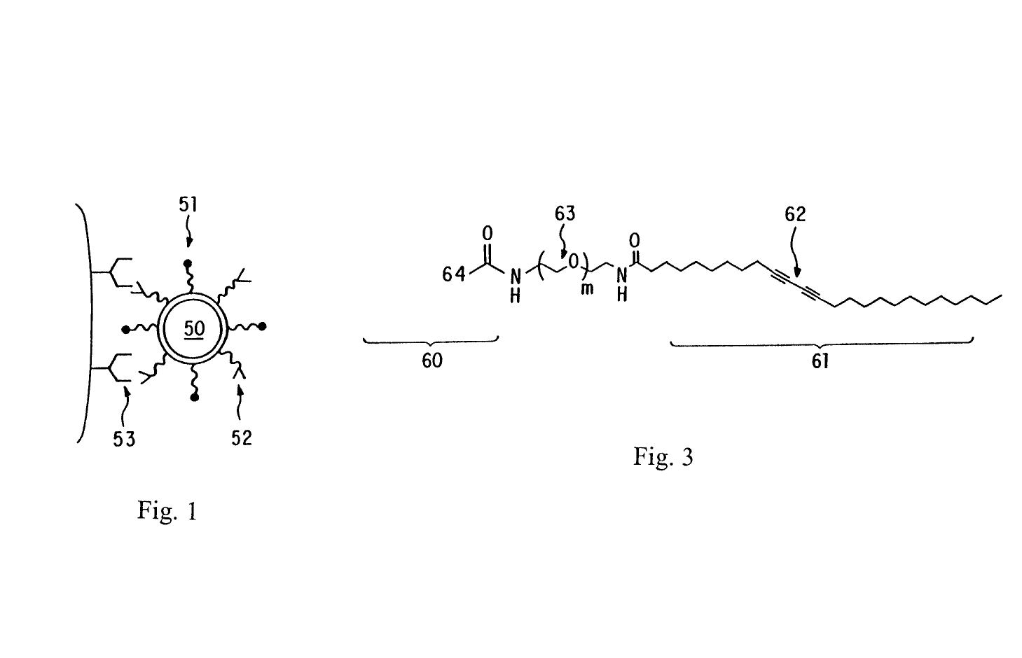

This invention relates to therapeutic and imaging agents which are comprised of a lipid construct, a targeting entity, and a therapeutic or treatment entity. FIG. 1 shows a schematic diagram of such a three-component system. The linking carrier 50 bears targeting entity 52 and therapeutic entity 51. Multiple copies of each targeting entity 52 and therapeutic entity 51 can be attached to each linking carrier 50. The targeting entity 52 serves to bind the entire vascular-targeted therapeutic agent to its target 53.

A “lipid construct,” as used herein, is a structure containing lipids, phospholipids, or derivatives thereof comprising a variety of different structural arrangements which lipids are known to adopt in aqueous suspension. These structures include, but are not limited to, lipid bilayer vesicles, micelles, liposomes, emulsions, lipid ribbons or sheets, and may be complexed with a variety of drugs and components which are known to be pharmaceutically acceptable. In the preferred embodiment, the lipid construct is a liposome. Common adjuvants include cholesterol and alpha-tocopherol, among others. The lipid constructs may be used alone or in any combination which one skilled in the art would appreciate to provide the characteristics desired for a particular application. In addition, the technical aspects of lipid construct, vesicle, and liposome formation are well known in the art and any of the methods commonly practiced in the field may be used for the present invention. The therapeutic or treatment entity may be associated with the agent by covalent or non-covalent means. As used herein, associated means attached to by covalent or noncovalent interactions.

Therapeutic Entities

The term “therapeutic entity” refers to any molecule, molecular assembly or macromolecule that has a therapeutic effect in a treated subject, where the treated subject is an animal, preferably a mammal, more preferably a human. The term “therapeutic effect” refers to an effect which reverses a disease state, arrests a disease state, slows the progression of a disease state, ameliorates a disease state, relieves symptoms of a disease state, or has other beneficial consequences for the treated subject. Therapeutic entities include, but are not limited to, drugs, such as doxorubicin and other chemotherapy agents; small molecule therapeutic drugs, toxins such as ricin; radioactive isotopes; genes encoding proteins that exhibit cell toxicity, and prodrugs (drugs which are introduced into the body in inactive form and which are activated in situ). Radioisotopes useful as therapeutic entities are described in Kairemo, et al., Acta Oncol. 35:343-55 (1996), and include Y-90, I-123, I-125, I-131, Bi-213, At-211, Cu-67, Sc-47, Ga-67, Rh-105, Pr-142, Nd-147, Pm-151, Sm-153, Ho-166, Gd-159, Tb-161, Eu-152, Er-171, Re-186, and Re-188.

Liposomes

As used herein, lipid refers to an agent exhibiting amphipathic characteristics causing it to spontaneously adopt an organized structure in water wherein the hydrophobic portion of the molecule is sequestered away from the aqueous phase. A lipid in the sense of this invention is any substance with characteristics similar to those of fats or fatty materials. As a rule, molecules of this type possess an extended apolar region and, in the majority of cases, also a water-soluble, polar, hydrophilic group, the so-called head-group. Phospholipids are lipids which are the primary constituents of cell membranes. Typical phospholipid hydrophilic groups include phosphatidylcholine and phosphatidylethanolamine moieties, while typical hydrophobic groups include a variety of saturated and unsaturated fatty acid moieties, including diacetylenes. Mixture of a phospholipid in water causes spontaneous organization of the phospholipid molecules into a variety of characteristic phases depending on the conditions used. These include bilayer structures in which the hydrophilic groups of the phospholipids interact at the exterior of the bilayer with water, while the hydrophobic groups interact with similar groups on adjacent molecules in the interior of the bilayer. Such bilayer structures can be quite stable and form the principal basis for cell membranes.

Bilayer structures can also be formed into closed spherical shell-like structures which are called vesicles or liposomes. The liposomes employed in the present invention can be prepared using any one of a variety of conventional liposome preparatory techniques. As will be readily apparent to those skilled in the art, such conventional techniques include sonication, chelate dialysis, homogenization, solvent infusion coupled with extrusion, freeze-thaw extrusion, microemulsification, as well as others. These techniques, as well as others, are discussed, for example, in U.S. Pat. No. 4,728,578, U.K. Patent Application G.B. 2193095 A, U.S. Pat. No. 4,728,575, U.S. Pat. No. 4,737,323, International Application PCT/US85/01161, Mayer et al., Biochimica et Biophysica Acta, Vol. 858, pp. 161-168 (1986), Hope et al., Biochimica et Biophysica Acta, Vol. 812, pp. 55-65 (1985), U.S. Pat. No. 4,533,254, Mahew et al., Methods In Enzymology, Vol. 149, pp. 64-77 (1987), Mahew et al., Biochimica et Biophysica Acta, Vol. 75, pp. 169-174 (1984), and Cheng et al., Investigative Radiology, Vol. 22, pp. 47-55 (1987), and U.S. Ser. No. 428,339, filed Oct. 27, 1989. The disclosures of each of the foregoing patents, publications and patent applications are incorporated by reference herein, in their entirety. A solvent free system similar to that described in International Application PCT/US85/01161, or U.S. Ser. No. 428,339, filed Oct. 27, 1989, may be employed in preparing the liposome constructions. By following these procedures, one is able to prepare liposomes having encapsulated therein a gaseous precursor or a solid or liquid contrast enhancing agent.

The materials which may be utilized in preparing the liposomes of the present invention include any of the materials or combinations thereof known to those skilled in the art as suitable in liposome construction. The lipids used may be of either natural or synthetic origin. Such materials include, but are not limited to, lipids such as cholesterol, phosphatidylcholine, phosphatidylethanolamine, phosphatidylserine, phosphatidylglycerol, phosphatidic acid, phosphatidylinositol, lysolipids, fatty acids, sphingomyelin, glycosphingolipids, glucolipids, glycolipids, sulphatides, lipids with amide, ether, and ester-linked fatty acids, polymerizable lipids, and combinations thereof. As one skilled in the art will recognize, the liposomes may be synthesized in the absence or presence of incorporated glycolipid, complex carbohydrate, protein or synthetic polymer, using conventional procedures. The surface of a liposome may also be modified with a polymer, such as, for example, with polyethylene glycol (PEG), using procedures readily apparent to those skilled in the art. Lipids may contain functional surface groups for attachment to a metal, which provides for the chelation of radioactive isotopes or other materials that serve as the therapeutic entity. Any species of lipid may be used, with the sole proviso that the lipid or combination of lipids and associated materials incorporated within the lipid matrix should form a bilayer phase under physiologically relevant conditions. As one skilled in the art will recognize, the composition of the liposomes may be altered to modulate the biodistribution and clearance properties of the resulting liposomes.

The membrane bilayers in these structures typically encapsulate an aqueous volume, and form a permeability barrier between the encapsulated volume and the exterior solution. Lipids dispersed in aqueous solution spontaneously form bilayers with the hydrocarbon tails directed inward and the polar headgroups outward to interact with water. Simple agitation of the mixture usually produces multilamellar vesicles (MLVs), structures with many bilayers in an onion-like form having diameters of 1-10 μm (1000-10,000 nm). Sonication of these structures, or other methods known in the art, leads to formation of unilamellar vesicles (UVs) having an average diameter of about 30-300 nm. However, the range of 50 to 200 nm is considered to be optimal from the standpoint of, e.g., maximal circulation time in vivo. The actual equilibrium diameter is largely determined by the nature of the phospholipid used and the extent of incorporation of other lipids such as cholesterol. Standard methods for the formation of liposomes are known in the art, for example, methods for the commercial production of liposomes are described in U.S. Pat. No. 4,753,788 to Ronald C. Gamble and U.S. Pat. No. 4,935,171 to Kevin R. Bracken.

Either as MLVs or UVs, liposomes have proven valuable as vehicles for drug delivery in animals and in humans. Active drugs, including small hydrophilic molecules and polypeptides, can be trapped in the aqueous core of the liposome, while hydrophobic substances can be dissolved in the liposome membrane. Other molecules, such as DNA or RNA, may be attached to the outside of the liposome for gene therapy applications. The liposome structure can be readily injected and form the basis for both sustained release and drug delivery to specific cell types, or parts of the body. MLVs, primarily because they are relatively large, are usually rapidly taken up by the reticuloendothelial system (the liver and spleen). The invention typically utilizes vesicles which remain in the circulatory system for hours and break down after internalization by the target cell. For these requirements the formulations preferably utilize UVs having a diameter of less than 200 nm, preferably less than 100 mn.

Linking Carriers

The term “linking carrier” refers to any entity which A) serves to link the therapeutic entity and the targeting entity, and B) confers additional advantageous properties to the vascular-targeted therapeutic agents other than merely keeping the therapeutic entity and the targeting entity in close proximity. Examples of these additional advantages include, but are not limited to: 1) multivalency, which is defined as the ability to attach either i) multiple therapeutic entities to the vascular-targeted therapeutic agents (i.e., several units of the same therapeutic entity, or one or more units of different therapeutic entities), which increases the effective “payload” of the therapeutic entity delivered to the targeted site; ii) multiple targeting entities to the vascular-targeted therapeutic agents (i.e., one or more units of different therapeutic entities, or, preferably, several units of the same targeting entity); or iii) both items i) and ii) of this sentence; and 2) improved circulation lifetimes, which can include tuning the size of the particle to achieve a specific rate of clearance by the reticuloendothelial system. The effective payload of therapeutic entity is the number of therapeutic entities delivered to the target site per binding event of the agent to the target. The payload will depend on the particular therapeutic entity and target. In some cases the payload will be as little as about 1 molecule delivered per binding event of the agent. In the case of a metal ion, the payload can be about one to 10³ molecules delivered per binding event. It is contemplated that the payload can be as high as 10⁴ molecules delivered per binding event. The payload can vary between about 1 to about 10⁴ molecules per binding event.

Preferred linking carriers are biocompatible polymers (such as dextran) or macromolecular assemblies of biocompatible components (such as liposomes). Examples of linking carriers include, but are not limited to, liposomes, polymerized liposomes, other lipid vesicles, dendrimers, polyethylene glycol assemblies, capped polylysines, poly(hydroxybutyric acid), dextrans, and coated polymers. A preferred linking carrier is a polymerized liposome. Polymerized liposomes are described in U.S. Pat. No. 5,512,294. Another preferred linking carrier is a dendrimer.

The linking carrier can be coupled to the targeting entity and the therapeutic entity by a variety of methods, depending on the specific chemistry involved. The coupling can be covalent or non-covalent. A variety of methods suitable for coupling of the targeting entity and the therapeutic entity to the linking carrier can be found in Hermanson, “Bioconjugate Techniques”, Academic Press: New York, 1996; and in “Chemistry of Protein Conjugation and Cross-linking” by S. S. Wong, CRC Press, 1993. Specific coupling methods include, but are not limited to, the use of bifunctional linkers, carbodiimide condensation, disulfide bond formation, and use of a specific binding pair where one member of the pair is on the linking carrier and another member of the pair is on the therapeutic or targeting entity, e.g. a biotin-avidin interaction.

Polymerized liposomes are self-assembled aggregates of lipid molecules which offer great versatility in particle size and surface chemistry. Polymerized liposomes are described in U.S. Pat. Nos. 5,512,294 and 6,132,764, incorporated by reference herein in their entirety. The hydrophobic tail groups of polymerizable lipids are derivatized with polymerizable groups, such as diacetylene groups, which irreversibly cross-link, or polymerize, when exposed to ultraviolet light or other radical, anionic or cationic, initiating species, while maintaining the distribution of functional groups at the surface of the liposome. The resulting polymerized liposome particle is stabilized against fusion with cell membranes or other liposomes and stabilized towards enzymatic degradation. The size of the polymerized liposomes can be controlled by extrusion or other methods known to those skilled in the art. Polymerized liposomes may be comprised of polymerizable lipids, but may also comprise saturated and non-alkyne, unsaturated lipids. The polymerized liposomes can be a mixture of lipids which provide different functional groups on the hydrophilic exposed surface. For example, some hydrophilic head groups can have functional surface groups, for example, biotin, amines, cyano, carboxylic acids, isothiocyanates, thiols, disulfides, α-halocarbonyl compounds, α,β-unsaturated carbonyl compounds and alkyl hydrazines. These groups can be used for attachment of targeting entities, such as antibodies, ligands, proteins, peptides, carbohydrates, vitamins, nucleic acids or combinations thereof for specific targeting and attachment to desired cell surface molecules, and for attachment of therapeutic entities, such as drugs, nucleic acids encoding genes with therapeutic effect or radioactive isotopes. Other head groups may have an attached or encapsulated therapeutic entity, such as, for example, antibodies, hormones and drugs for interaction with a biological site at or near the specific biological molecule to which the polymerized liposome particle attaches. Other hydrophilic head groups can have a functional surface group of diethylenetriamine pentaacetic acid, ethylenedinitrile tetraacetic acid, tetraazocyclododecane-1, 4, 7, 10-tetraacetic acid (DOTA), porphoryin chelate and cyclohexane-1,2,-diamino-N, N′-diacetate, as well as derivatives of these compounds, for attachment to a metal, which provides for the chelation of radioactive isotopes or other materials that serve as the therapeutic entity. Examples of lipids with chelating head groups are provided in U.S. Pat. No. 5,512,294, incorporated by reference herein in its entirety.

Large numbers of therapeutic entities may be attached to one polymerized liposome that may also bear from several to about one thousand targeting entities for in vivo adherence to targeted surfaces. The improved binding conveyed by multiple targeting entities can also be utilized therapeutically to block cell adhesion to endothelial receptors in vivo. Blocking these receptors can be useful to control pathological processes, such as inflammation and control of metastatic cancer. For example, multi-valent sialyl Lewis X derivatized liposomes can be used to block neutrophil binding, and antibodies against VCAM-1 on polymerized liposomes can be used to block lymphocyte binding, e.g. T-cells.

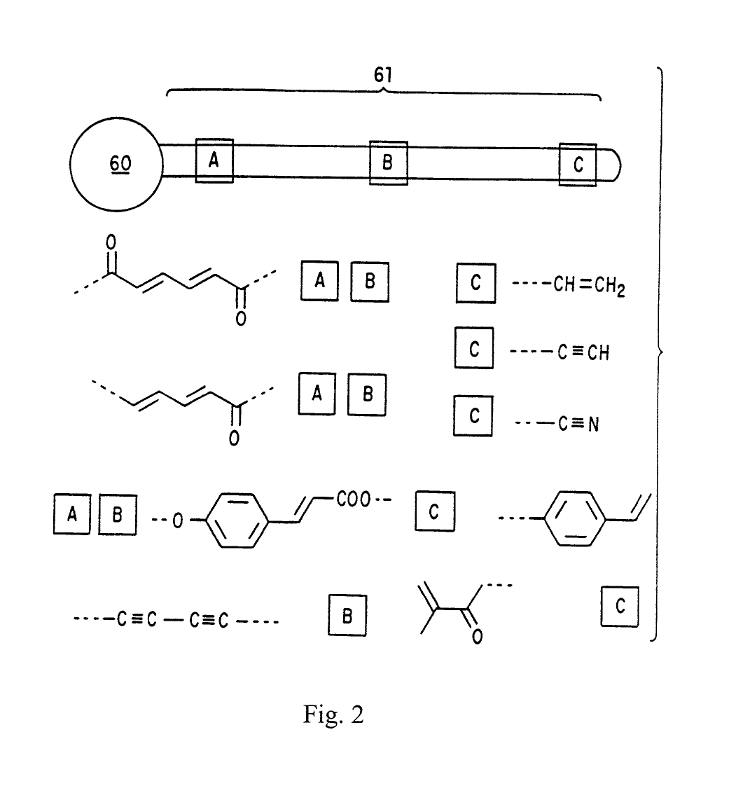

FIGS. 2 and 3 schematically show a polymerizable lipid molecule for use in making polymerized liposomes. The amphiphilic lipid molecule has a polar head group 60 and a hydrophobic tail group 61. The tail portion of the lipid has a polymerizable functional group 62, such as diacetylene, olefins, acetylenes, nitrites, alkyl styrenes, esters, thiols, amides and alpha, beta unsaturated carbonyl compounds forming liposomes that will polymerize upon irradiation by an electromagnetic source, such as UV light, or by chemical or thermal means. FIG. 2 shows polymerizable functional groups which may be located at specific positions A, B and C on tail group 61. As shown in FIG. 3, the head group and tail group are joined by variable length spacer portion 63. The length of the spacer portion, indicated by m, controls the distance of the active agent from the surface of the particle to make it more available for its active function. The spacer portion may be a bifunctional aliphatic compounds which can include heteroatoms or bifunctional aromatic compounds. Preferred spacer portions are compounds such as, for example, variable length polyethylene glycol, polypropylene glycol, polyglycine, bifunctional aliphatic compounds, for example amino caproic acid, or bifunctional aromatic compounds. The head group has a functional surface group 64, such as diethylenetriamine pentaacetic acid (DTPA), isothiocyanato-diethylenetriamine pentaacetic acid ITC-DTPA), ethylenedinitrile tetraacetic acid (EDTA), tetraazocyclododecane 1, 4, 7, 10-tetraacetic acid (DOTA), cyclohexane-1,2-diamino-N, N′-diacetate (CHTA), MX-DTPA (isothiocyanato-benzyl-methyl-diethylenetriaminepentaacetic acid) or citrate, for chelating a metal, or biotin, amines, carboxylic acids and alkyl hydrazines for coupling biologically active targeting agents, such as ligands, antibodies, peptides or carbohydrates for specific cell surface receptors or antigenic determinants.

Generally, lipids suitable for use in polymerized liposomes have an active head group for attaching a therapeutic entity or targeting entity, a spacer portion for accessibility of the active head group; a hydrophobic tail for self-assembly into liposomes; and a polymerizable group to stabilize the liposomes.

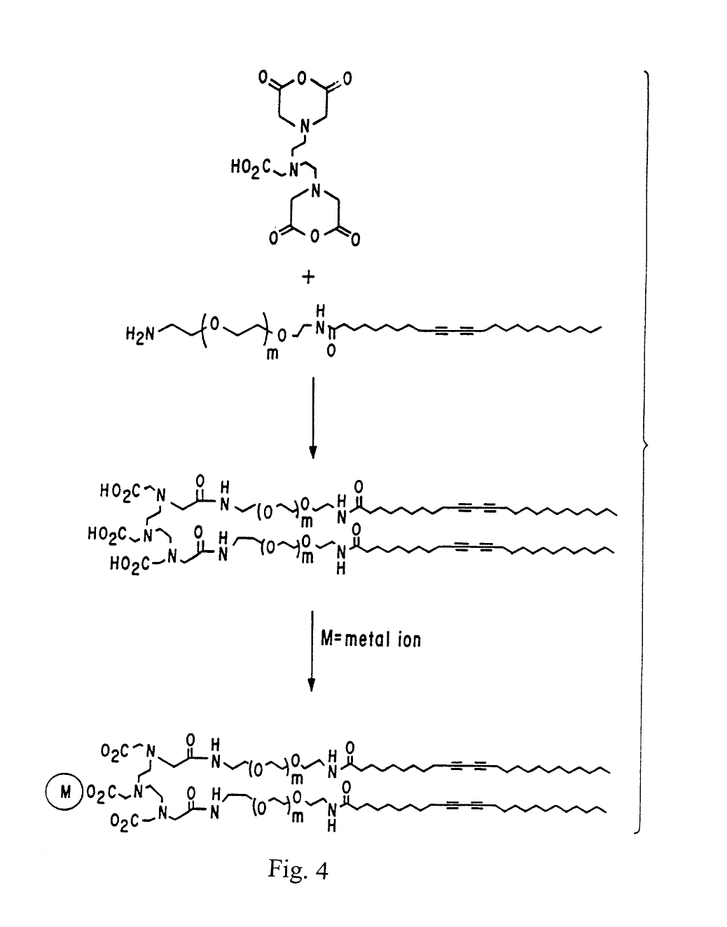

A unique lipid is synthesized containing pentacosadiynoic acid conjugated to diethylenetriamine pentaacetic acid via a variable length polyethylene glycol spacer as shown in FIG. 4. These amphipathic molecules have metal chelates as head groups connected to a lipid tail which contains a polymerizable diacetylene moiety. The spacer length can be controlled by the choice of commercially available variable length polyethylene glycol derivatives.

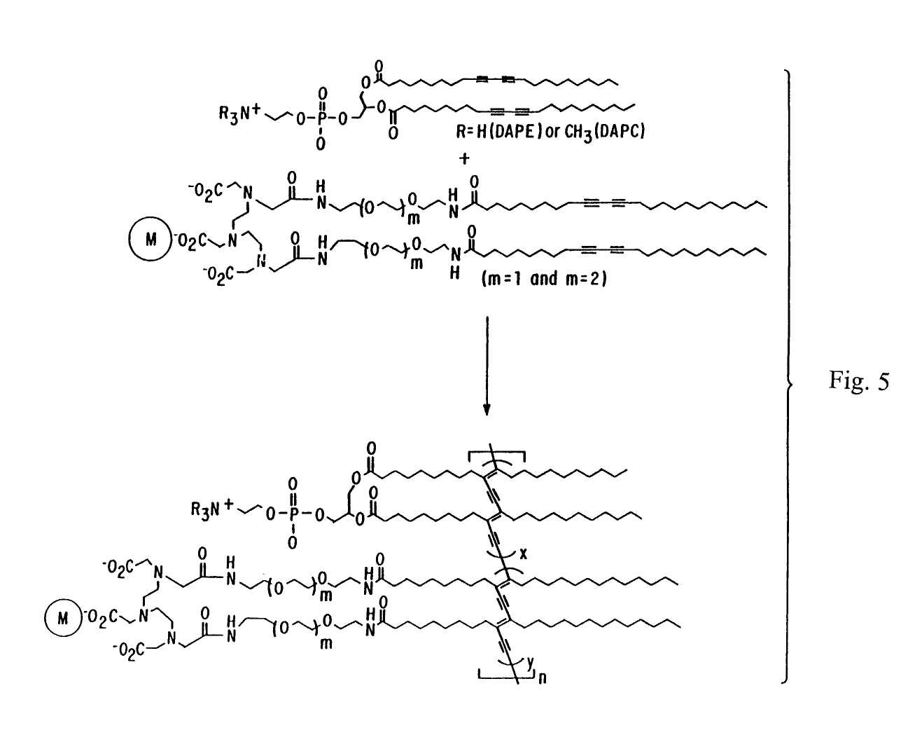

Specifically, compounds such as the one shown in FIG. 4 are synthesized by reacting the NHS ester of the lipid pentacosadiynoic acid (PDA) with triethyleneglycol-diamine and tetraethyleneglycol-diamine spacers to form the corresponding PEGₘ-PDA amides, m=1 or 2, then reacting the PEGₘ-PDA amide with diethylenetriamine pentaacetic acid dianhydride (DTPAA) to form diethylenetriamine pentaacetic acid-bis(tri or tetraethylene glycol-pentacosadiynoic acid) diamide (DTPA-bis-(PEGₘ-PDA), m=1 or 2 diamide). The diamide is then treated with a metal ion source M, such as gadolinium trichloride, dysprosium trichloride or a technicium or indium derivative to form the amphiphilic metal chelate as shown in FIG. 4 with a polyethylene spacer (m 1 and m=2). The diamide-lanthanide chelate, shown in FIG. 4 and as a reactant in FIG. 5, is mixed with a matrix lipid of diacetylenic choline (DAPC, R=CH₃) or diacetylenic ethanolamine (R=H), shown in FIG. 5, pentacosadiynoic acid (PDA) or derivatives of PDA in an amount to result in the desired surface density of metal on the polymerized liposomes. The matrix lipid forms polymerizable liposomes under a variety of conditions and closely mimics the topology of in vivo cell membranes.

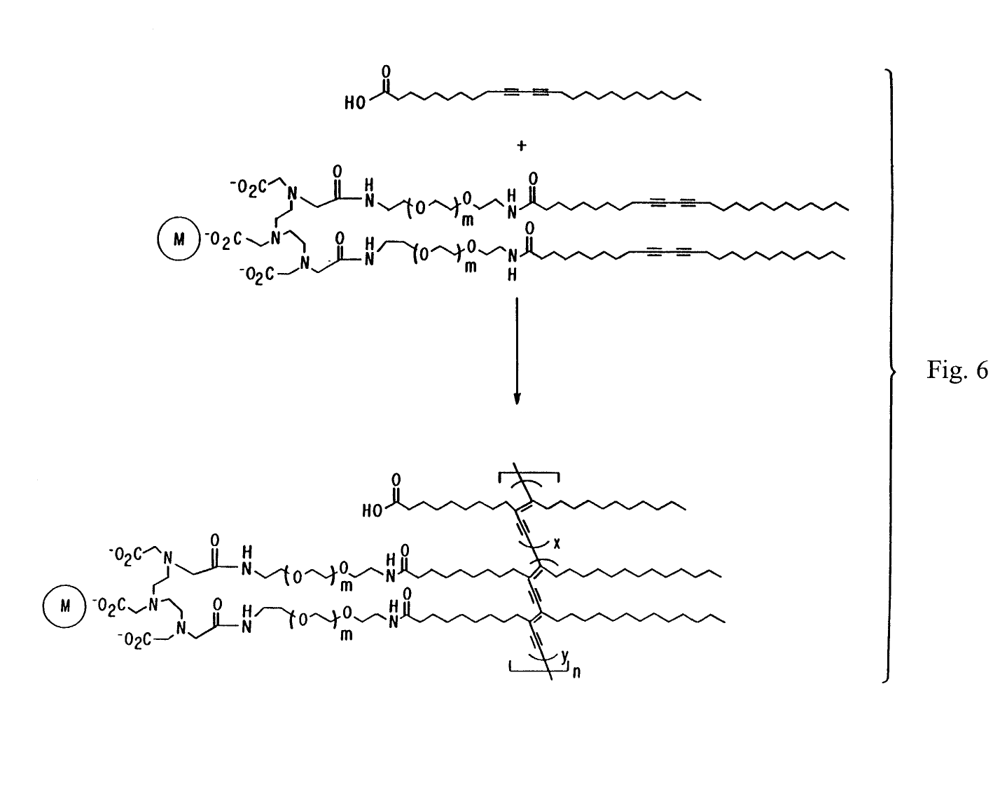

To form the polymerized liposome shown as the product in FIGS. 5 and 6, the metal chelated diamide shown in FIG. 4 is doped into the DAPC, as shown in FIG. 5, or PDA, as shown in FIG. 6, matrix in organic solvent. The organic solvent is evaporated and the dried lipid film is hydrated to a known lipid density, such as 15 mM total lipid, with the desired buffer or water. The resulting suspension is sonicated at temperatures above the gel-liquid crystal phase transition for DAPC or PDA, Tm=40° C., with a probe-tip sonicator. A nearly clear, colorless solution of emulsified vesicles, or liposomes, is produced. It was determined by transmission electron microscopy and atomic force microscopy that these liposomes are on average 20 to 200 nm in diameter. Their size can be reduced by extrusion at temperatures greater than Tm through polycarbonate filters with well defined porosity. The liposomes are polymerized by cooling the solution to 4° C. on a bed of ice and irradiating at 254 nm with a UV lamp. Alternatively, the liposomes can be irradiated at room temperature and then cooled while continuing UV irradiation. The resulting polymerized liposomes, diagrammatically shown as the products in FIGS. 5 and 6, are orange in color when using DAPC with two visible absorption bands centered at 490 nm and 510 nm arising from the conjugated ene-yne diacetylene polymer and generally blue in color when using PDA with absorption bands around 540 nm and 630 nm. These liposomes can undergo a blue to red transition when molecules bind to their surface after heating or resonication or after standing at room temperature for extended times or being treated with organic solvents. This transition may be useful for developing a detection system for these conditions.

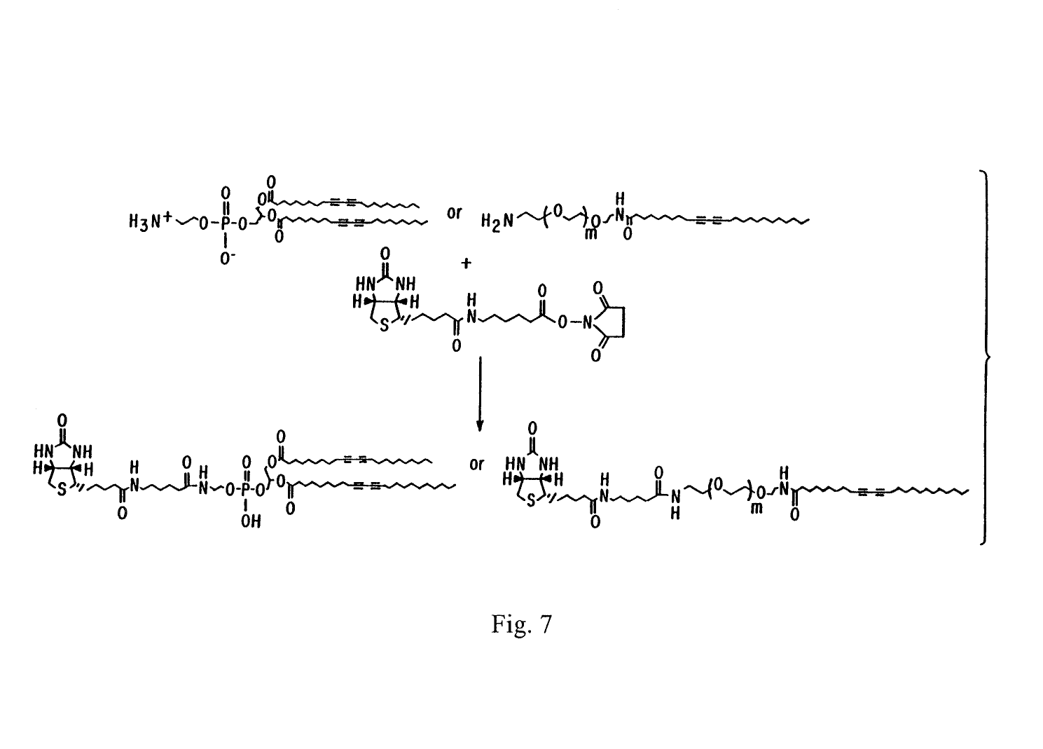

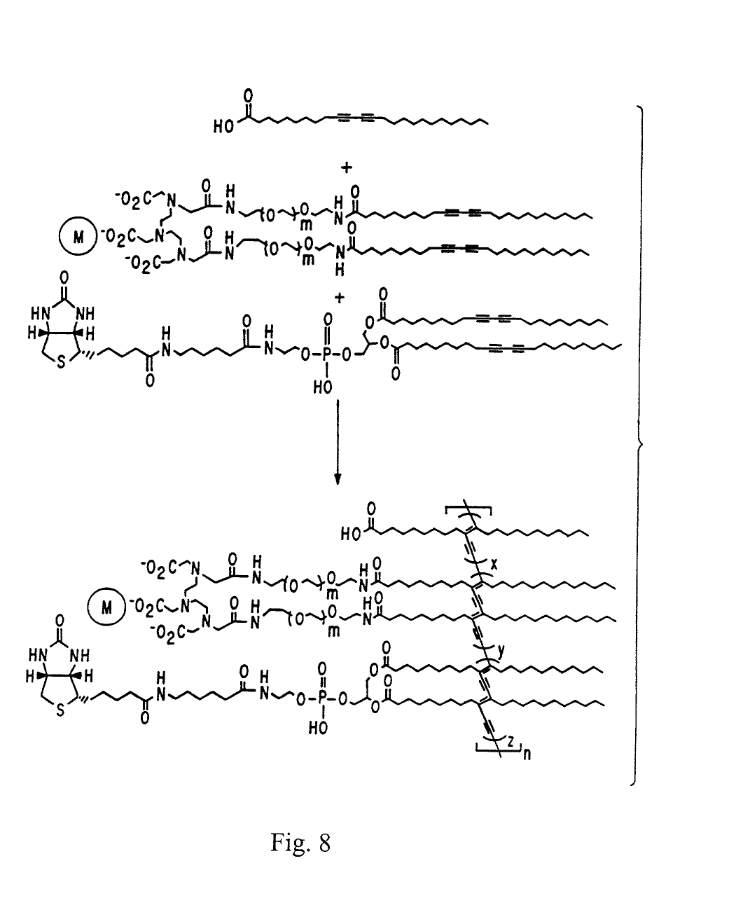

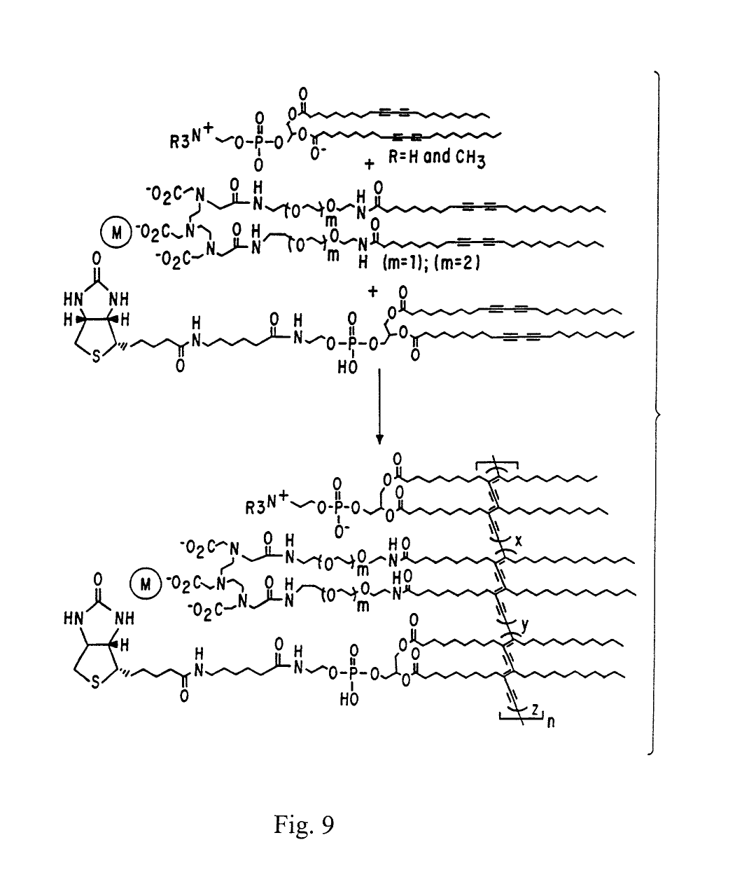

Targeted polymerized liposomes were produced from biotinylated or negatively charged liposomes to which biotinylated antibodies are attached through avidin, which has a high affinity for biotin and a high positive charge. In addition to biotin-avidin crosslinking, antibody-avidin conjugates can be attached to the polymerized liposome via charge-charge interactions similar to ion exchange. Commercially available diacetylene glycerophosphoethanolamine (DAPE) lipid is converted to its biotinylated analog by acylation of the amine terminated lipid with commercially available biotinylating agents, such as biotinamidocaproate N-hydroxysuccinimide ester or paranitrophenol esters, as shown in FIG. 7. The biotinylated polymerized liposomes are produced by incorporating the biotinylated lipid in a matrix of lipids of either PDA, DAPE or DAPC as shown in FIGS. 8 and 9, respectively. Negatively charged polymerized liposomes may be constructed by using pentacosadiynoic acid or other negatively charged lipid as a matrix lipid.

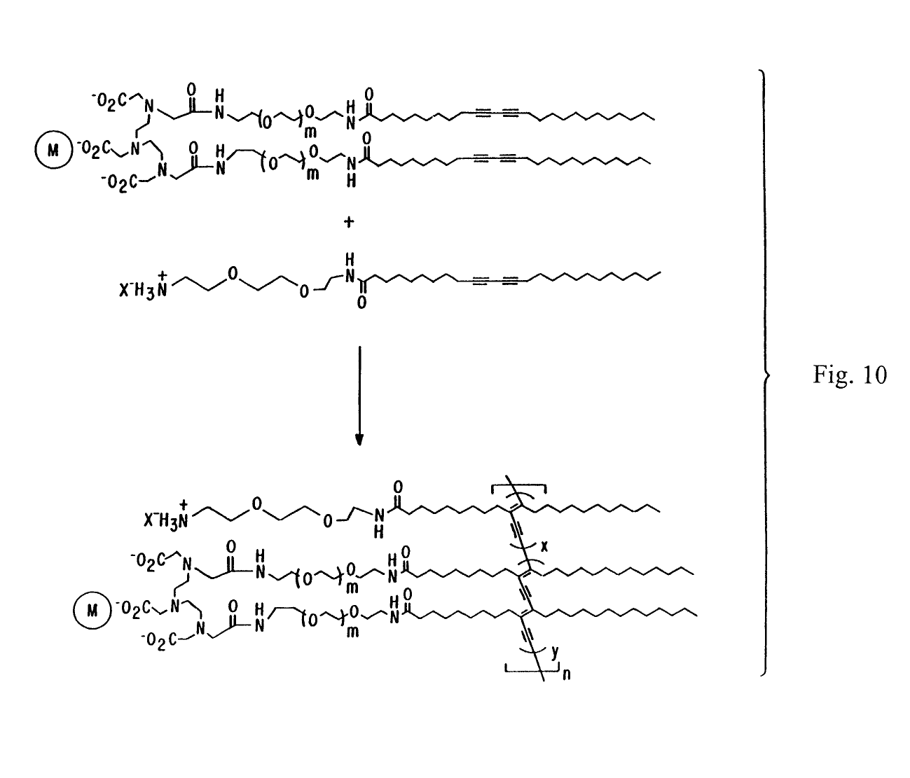

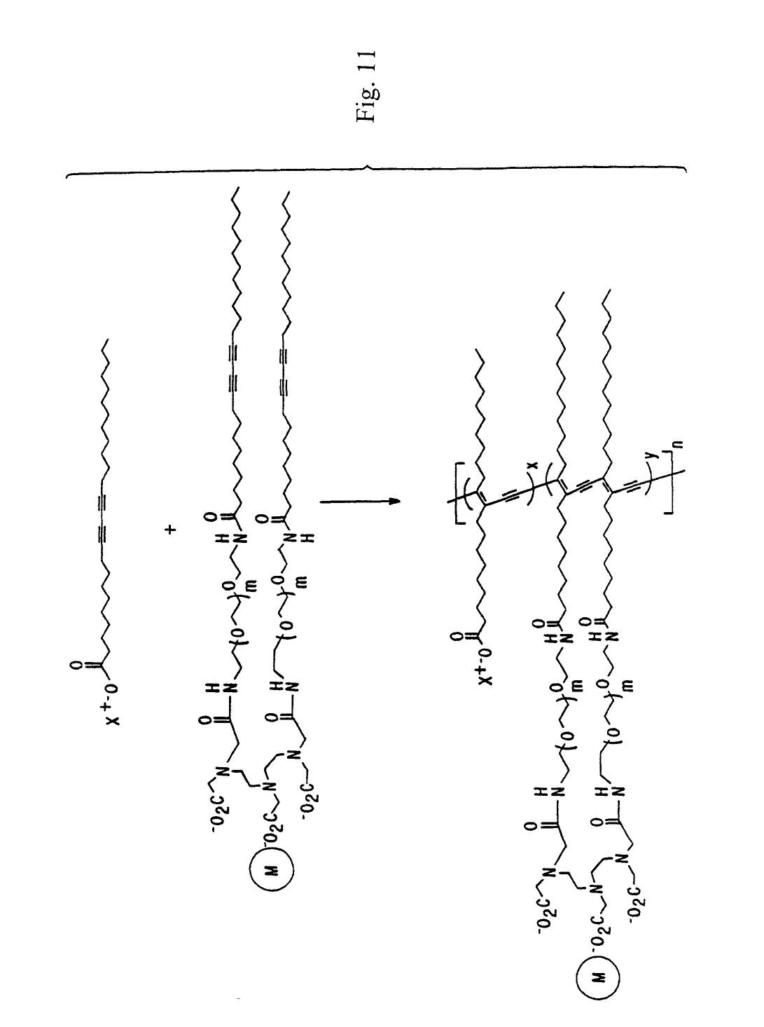

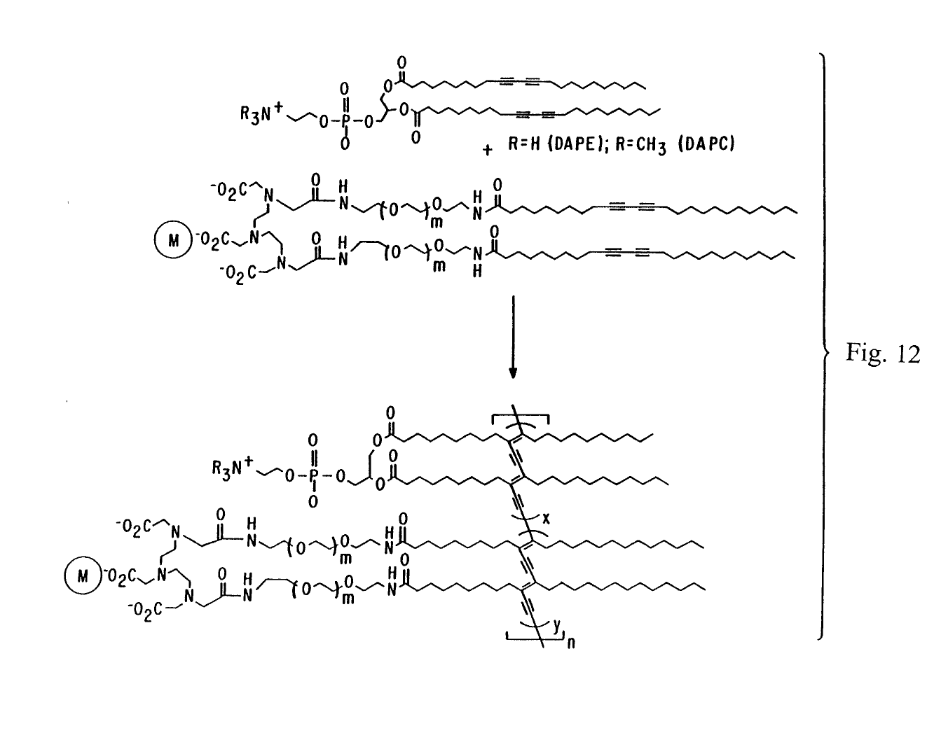

The liposomes useful herein include a broad based group of liposomes having varied functionality which includes liposomes containing positively charged groups, such as amines as shown in FIG. 10, negatively charged groups, such as carboxylates as shown in FIG. 11, and neutral groups, such as zwitterions as shown in FIG. 12. These groups are important to control biodistribution, blood pool half-life and non-specific adhesion of the particles.

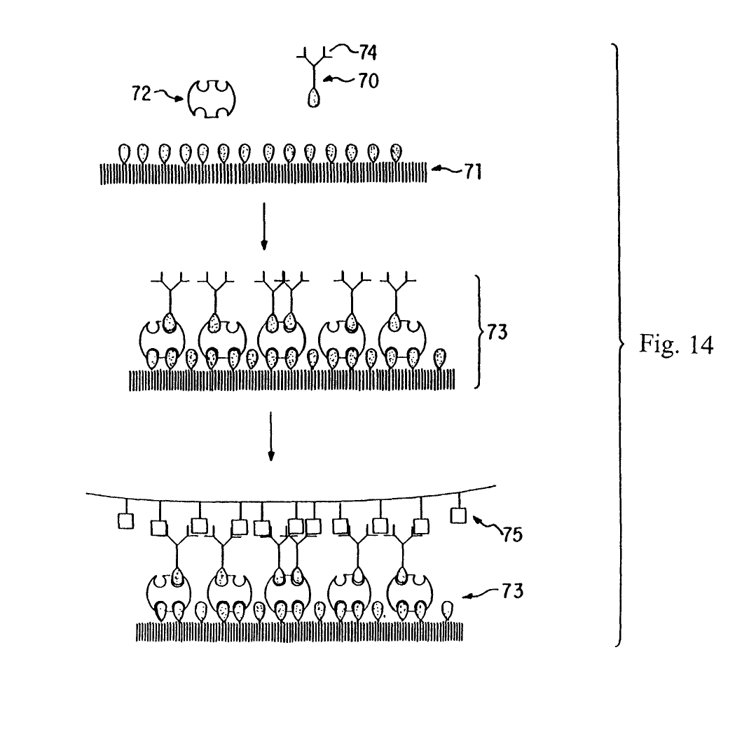

Biotinylated polymerized liposomes with a biotinylated anti-VCAM-1 antibody attached via a biotin avidin sandwich were produced in the manner described above. This targeted polymerized liposome binds to VCAM-1, a leukocyte adhesion receptor on the endothelial surface which is upregulated during inflammation. In vitro histology demonstrated specific interaction between the polymerized liposomes and the inflamed brainstem tissue from a mouse with allergic autoimmune encephalitis. The formation of such biotinylated antibody coated polymerized liposomes and their attachment to in vivo cell receptors is schematically shown in FIG. 14. As shown in FIG. 14, the biotinylated antibody 70 having functional group 74 is attached to the biotinylated lipid surface 71 through bridge 72 of avidin or streptavidin to form antibody-coated polymerized liposomes 73. The functional group 74 of antibody 70 is attached in vivo to an endothelium receptor 75, thereby attaching the polymerized liposome to the endothelium for external detection.

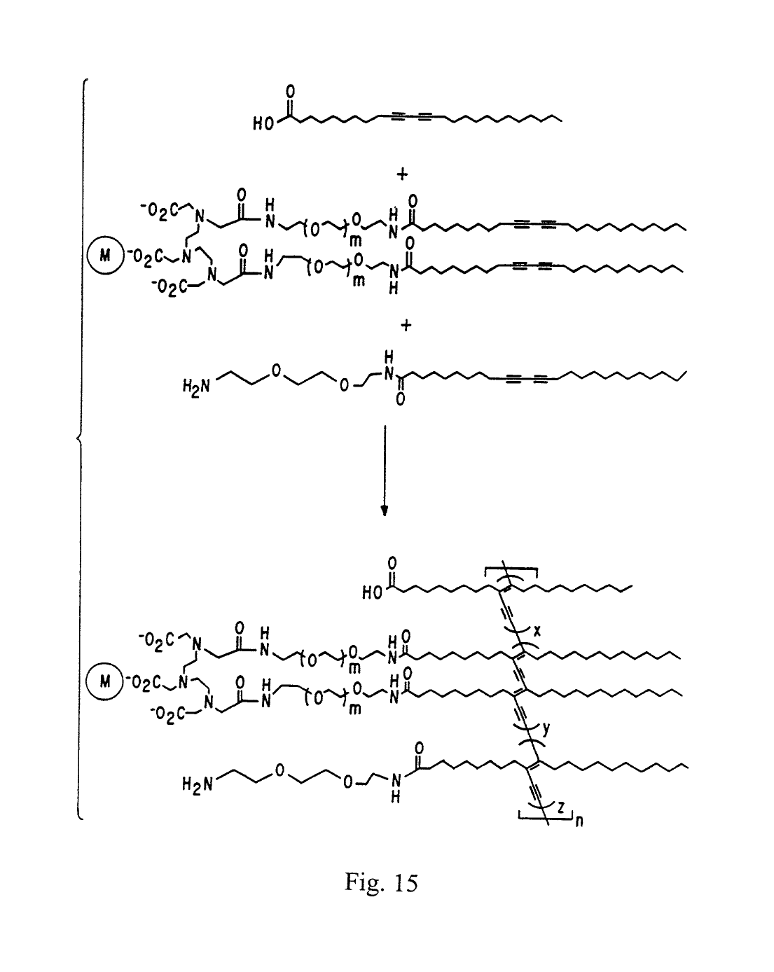

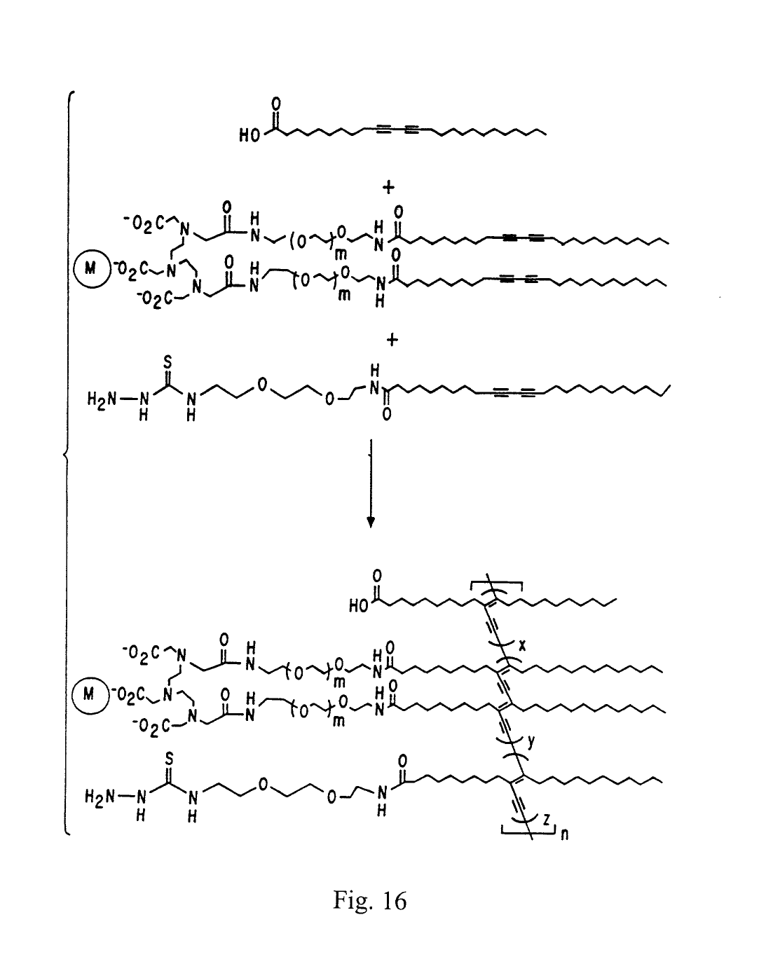

Antibodies may also be attached by “direct” methods. For example, the lipids comprising the liposome can contain a group, such as an amine or hydrazine derivative, that reacts with aldehydes on oxidized antibodies and oligosaccharides. Liposomes containing amine, FIG. 15, and hydrazine, FIG. 16, head groups have been constructed for this purpose. Antibodies can also be attached by charge-charge interaction such as ion exchange. In this case, the antibody is bound to a positively charged protein, such as, for example avidin and this complex ion may be exchanged onto negatively charged polymerized liposomes.

Antibody-conjugated polymerized liposomes achieve in vitro and in vivo targeting of specific molecules associated with specific body tissues and specific molecules associated with specific bodily functions and pathologies. This has been demonstrated by using MRI contrast agents on the targeted polymerized liposomes, which has provided direct evidence of the biodistribution of the targeted polymerized liposomes. The polymerized liposomes are thus suitable for targeted delivery of drugs for therapeutic treatments. Various therapeutic entities can be encapsulated or attached to the surface of polymerized liposomes for delivery to specific sites in vivo. By using target-specific drug-carrying polymerized liposomes which also carry a contrast enhancement agent, the drug delivery can be simultaneously visualized by magnetic resonance imaging.

Targeted polymerized liposomes which recirculate in the vasculature may include endothelial antigens which interact with the cell adhesion molecules or other cell surface receptors to retain a number of the targeted polymerized liposomes at the desired location. The high concentration of therapeutic entities in the polymerized liposomes render possible site-specific delivery of high concentrations of drugs or other therapeutic entities, while minimizing the burden on other tissues. The polymerized liposomes described herein are particularly well-suited since they maintain their integrity in vivo, recirculate in the blood pool, are rigid and do not easily fuse with cell membranes, and serve as a scaffold for attachment of both the antibodies/targeting entities and the therapeutic entities. The size distribution, particle rigidity and surface characteristics of the polymerized liposomes can be tailored to avoid rapid clearance by the reticuloendothelial system and the surface can be modified with ethylene glycol to further increase intravascular recirculation times. In one embodiment, the polymerized liposomes were found to have blood pool half-lives of about 20 hours in rats.

In one embodiment, the site-specific polymerized liposomes having attached monoclonal antibodies for specific receptor targeting may be used to deliver therapeutic entities to cells expressing intercellular adhesion molecule-1, ICAM-1. This marker is upregulated in murine experimental autoimmune encephalitis, an animal model for multiple sclerosis.

Another preferred linking carrier is a dendrimer. Dendrimers are polymers with well-defined branching from a central core (e.g., “starburst polymers”). In contrast to conventional polymers, dendrimers tend to be highly branched, monodisperse macromolecules, i.e., the molecular weight tends to be very well-defined instead of a range as with conventional linear or branched polymers. Dendrimers are described in U.S. Pat. Nos. 4,507,466, 4,558,120, 4,568,737, 4,587,329, 4,631,337, 4,694,064, 4,737,550, and 4,857,599, as well as numerous other patents and patent publications. Dendrimer structure, synthesis, and characteristics are reviewed in Kim and Zimmerman, “Applications of dendrimers in bio-organic chemistry,” Current Opinion In Chemical Biology (1998) 2(6):733-42; Tam and Spetzler, “Chemoselective approaches to the preparation of peptide dendrimers and branched artificial proteins using unprotected peptides as building blocks,” Biomedical Peptides, Proteins & Nucleic Acids (1995) 1(3):123-32; Frechet, “Functional polymers and dendrimers: reactivity, molecular architecture, and interfacial energy,” Science (1994) 263(5154):1710-5; Liu and Frechet, “Designing dendrimers for drug delivery,” Pharmaceutical Science and Technology Today (1999) 2(10):393401; Verprek and Jezek “Peptide and glycopeptide dendrimers. Part I,” Journal of Peptide Science (1999) 5(1):5-23; Veprek and Jezek, “Peptide and glycopeptide dendrimers. Part II,” Journal Of Peptide Science (1999) 5(5)203-20; Tomalia et al., “Starburst dendrimers: Molecular-level control of size, shape, surface chemistry, topology, and flexibility from atoms to macroscopic matter” Angewandte Chemie—International Edition in English (1990) 29(2):138-175; Bosman et al., “About dendrimers: Structure, physical properties, and applications” Chemical Reviews (1999) 99(7):1665-1688; Fischer and Vogtle, “Dendrimers: From design to application—A progress report,” Angewandte Chemie-International Edition (1999) 38(7):885905; Roovers and Comanita, “Dendrimers And Dendrimer-Polymer Hybrids,” Advances In Polymer Science (1999) 142:179-228; Smith and Diederich, “Functional Dendrimers: Unique Biological Mimics,” Chemistry—A European Journal (1998) 4(8):1353-1361; and Matthews et al., “Dendrimers—Branching out from curiosities into new technologies,” Progress In Polymer Science (1998) 23(1): 1-56. The synthesis of dendrimers typically uses reiterative synthetic cycles, allowing control over the dendrimer's size, shape, surface chemistry, flexibility, and interior topology. An example of a dendrimer suitable for use as a linking entity is described in Wu et al., “Metal-Chelate-Dendrimer-Antibody Constructs for Use in Radioimmunotherapy and Imaging,” Bioorganic and Medicinal Chemistry Letters (1994) 4(3):449-454.

Dendrimers can be readily used as linking carriers by employing a variety of chemical conjugation techniques to attach the targeting entity and therapeutic entity. For example, in U.S. Pat. No. 6,020,457, which discloses a dendrimer having a disulfide (—S—S—) bond in its core, the dendrimer can be constructed by the methods described in the patent. The final external layer of the dendrimer can be capped with a reactive group such as an amine or carboxyl group. These reactive groups can then be derivatized with either targeting entities or therapeutic entities (or, in some cases, a mixture of both). The core disulfide bond can then be reduced to a thiol, and the complementary entity attached via the thiol functionality. That is, if a therapeutic entity had been attached to the external layer of the dendrimeric linking carrier, upon reduction and formation of the thiol functionality, a targeting entity can be attached via the free —SH group. One example of such targeting entity is an N-terminal-iodoacetylated peptide (the peptide may be a hormone or bioactive fragment of a larger protein), which is readily synthesized by standard solid-phase peptide techniques. The iodoacetyl group will react with the free thiol functionality, resulting in the conjugation of the therapeutic-entity-derivatized linking carrier with the targeting entity (the peptide).

The polymerized liposome particle can also contain groups to control nonspecific adhesion and reticuloendothelial system uptake. For example, PEGylation of liposomes has been shown to prolong circulation lifetimes; see International Patent Application WO 90/04384.

The component lipids of polymerized liposomes can be purified and characterized individually using standard, known techniques and then combined in controlled fashion to produce the final particle. The polymerized liposomes can be constructed to mimic native cell membranes or present functionality, such as ethylene glycol derivatives, that can reduce their potential immunogenicity. Additionally, the polymerized liposomes have a well-defined bilayer structure that can be characterized by known physical techniques such as transmission electron microscopy and atomic force microscopy.

Stabilizing Entities

The agents of the present invention optionally contain a stabilizing entity. As used herein, “stabilizing entity” refers to a macromolecule or polymer, which may optionally contain chemical functionality for the association of the stabilizing entity to the surface of the vesicle, and/or for subsequent association of therapeutic entities or targeting agents. The polymer should be biocompatible. Polymers useful to stabilize the liposomes of the present invention may be of natural, semi-synthetic (modified natural) or synthetic origin. A number of stabilizing entities which may be employed in the present invention are available, including xanthan gum, acacia, agar, agarose, alginic acid, alginate, sodium alginate, carrageenan, gelatin, guar gum, tragacanth, locust bean, bassorin, karaya, gum arabic, pectin, casein, bentonite, unpurified bentonite, purified bentonite, bentonite magma, and colloidal bentonite.

Other natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrose, dextrin, glucose, polyglucose, polydextrose, pustulan, chitin, agarose, keratin, chondroitin, dermatan, hyaluronic acid, alginic acid, xanthin gum, starch and various other natural homopolyner or heteropolymers, such as those containing one or more of the following aldoses, ketoses, acids or amines: erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Other suitable polymers include proteins, such as albumin, polyalginates, and polylactide-glycolide copolymers, cellulose, cellulose (microcrystalline), methylcellulose, hydroxyethylcellulose, hydroxypropylcellulose, hydroxypropylmethylcellulose, carboxymethylcellulose, and calcium carboxymethylcellulose.

Exemplary semi-synthetic polymers include carboxymethylcellulose, sodium carboxymethylcellulose, carboxymethylcellulose sodium 12, hydroxymethylcellulose, hydroxypropylmethylcellulose, methylcellulose, and methoxycellulose. Other semi-synthetic polymers suitable for use in the present invention include carboxydextran, aminodextran, dextran aldehyde, chitosan, and carboxymethyl chitosan.

Exemplary synthetic polymers include poly(ethylene imine) and derivatives, polyphosphazenes, hydroxyapatites, fluoroapatite polymers, polyethylenes (such as, for example, polyethylene glycol, the class of compounds referred to as Pluronics®, commercially available from BASF, (Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof, polysorbate, carbomer 934P, magnesium aluminum silicate, aluminum monostearate, polyethylene oxide, polyvinylalcohol, povidone, polyethylene glycol, and propylene glycol. Methods for the preparation of vesicles which employ polymers to stabilize vesicle compositions will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

In a preferred embodiment, the stabilizing entity is dextran. In another preferred embodiment, the stabilizing entity is a modified dextran, such as amino dextran. Without being bound by theory, it is believed that dextran may increase circulation times of liposomes in a manner similar to PEG. In other preferred embodiments, the following polymers and their derivatives are used. poly(galacturonic acid), poly(L-glutamic acid), poly(L-glutamic acid-L-tyrosine), poly[R)-3-hydroxybutyric acid], poly(inosinic acid potassium salt), poly(L-lysine), poly(acrylic acid), poly(ethanolsulfonic acid sodium salt), poly(methylhydrosiloxane), poly(vinyl alcohol), poly(vinylpolypyrrolidone), poly(vinylpyrrolidone), poly(glycolide), poly(lactide), poly(lactide-co-glycolide), and hyaluronic acid. In other preferred embodiments, copolymers including a monomer having at least one reactive site, and preferably multiple reactive sites, for the attachment of the copolymer to the vesicle or other molecule.

In some embodiments, the polymer may act as a hetero- or homobifunctional linking agent for the attachment of targeting agents, therapeutic entities, or chelators such as DTPA and its derivatives.

In one embodiment, the stabilizing entity is associated with the vesicle by covalent means. In another embodiment, the stabilizing entity is associated with the vesicle by non-covalent means. Covalent means for attaching the targeting entity with the liposome are known in the art and described in the EXAMPLES section.

Noncovalent means for attaching the targeting entity with the liposome include but are not limited to attachment via ionic, hydrogen-bonding interactions, including those mediated by water molecules or other solvents, hydrophobic interactions, or any combination of these.

In a preferred embodiment, the stabilizing agent forms a coating on the liposome, polymerized liposome, or other linking carrier.

Targeting Entities

The term “targeting entity” refers to a molecule, macromolecule, or molecular assembly which binds specifically to a biological target. Examples of targeting entities include, but are not limited to, antibodies (including antibody fragments and other antibody-derived molecules which retain specific binding, such as Fab, F(ab′)2, Fv, and scFv derived from antibodies); receptor-binding ligands, such as hormones or other molecules that bind specifically to a receptor; cytokines, which are polypeptides that affect cell function and modulate interactions between cells associated with immune, inflammatory or hematopoietic responses; molecules that bind to enzymes, such as enzyme inhibitors; nucleic acid ligands or aptamers, and one or more members of a specific binding interaction such as biotin or iminobiotin and avidin or streptavidin. Preferred targeting entities are molecules which specifically bind to receptors or antigens found on vascular cells. More preferred are molecules which specifically bind to receptors, antigens or markers found on cells of angiogenic neovasculature or receptors, antigens or markers associated with tumor vasculature. The receptors, antigens or markers associated with tumor vasculature can be expressed on cells of vessels which penetrate or are located within the tumor, or which are confined to the inner or outer periphery of the tumor. In one embodiment, the invention takes advantage of pre-existing or induced leakage from the tumor vascular bed; in this embodiment, tumor cell antigens can also be directly targeted with agents that pass from the circulation into the tumor interstitial volume.

Other targeting entities target endothelial receptors, tissue or other targets accessible through a body fluid or receptors or other targets upregulated in a tissue or cell adjacent to or in a bodily fluid. For example, targeting entities attached to carriers designed to deliver drugs to the eye can be injected into the vitreous, choroid, or sclera; or targeting agents attached to carriers designed to deliver drugs to the joint can be injected into the synovial fluid.

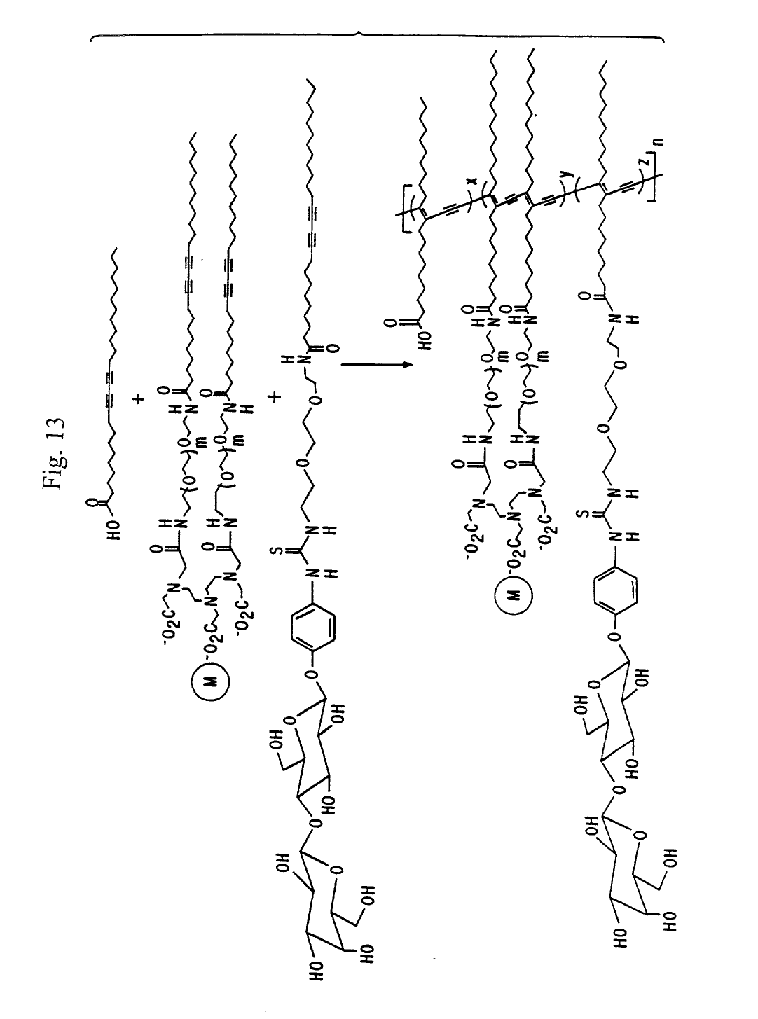

Targeting entities attached to the polymerized liposomes, or linking carriers of the invention include, but are not limited to, small molecule ligands, such as carbohydrates, and compounds such as those disclosed in U.S. Pat. No. 5,792,783 (small molecule ligands are defined herein as organic molecules with a molecular weight of about 1000 daltons or less, which serve as ligands for a vascular target or vascular cell marker); proteins, such as antibodies and growth factors; peptides, such as RGD-containing peptides (e.g. those described in U.S. Pat. No. 5,866,540), bombesin or gastrin-releasing peptide, peptides selected by phage-display techniques such as those described in U.S. Pat. No. 5,403,484, and peptides designed de novo to be complementary to tumor-expressed receptors; antigenic determinants; or other receptor targeting groups. These head groups can be used to control the biodistribution, non-specific adhesion, and blood pool half-life of the polymerized liposomes. For example, β-D-lactose has been attached on the surface, as shown in FIG. 13, to target the asialoglycoprotein (ASG) found in liver cells which are in contact with the circulating blood pool. Glycolipids can be derivatized for use as targeting entities by converting the commercially available lipid (DAGPE) or the PEG-PDA amine shown in FIG. 4 into its isocyanate followed by treatment with triethylene glycol diamine spacer to produce the amine terminated thiocarbamate lipid which by treatment with the para-isothiocyanophenyl glycoside of the carbohydrate ligand produces the desired targeting glycolipids. This synthesis provides a water-soluble flexible spacer molecule spaced between the lipid that will form the internal structure or core of the liposome and the ligand that binds to cell surface receptors, allowing the ligand to be readily accessible to the protein receptors on the cell surfaces. The carbohydrate ligands can be derived from reducing sugars or glycosides, such as para-nitrophenyl glycosides, a wide range of which are commercially available or easily constructed using chemical or enzymatic methods. Polymerized liposomes coated with carbohydrate ligands can be produced by mixing appropriate amounts of individual lipids followed by sonication, extrusion and polymerization and filtration as described above and shown in FIG. 13. Suitable carbohydrate derivatized polymerized liposomes have about 1 to about 30 mole percent of the targeting glycolipid and filler lipid, such as PDA, DAPC or DAPE, with the balance being metal chelated lipid. Other lipids may be included in the polymerized liposomes to assure liposome formation and provide high contrast and recirculation.

In some embodiments, the targeting entity targets the liposomes to a cell surface. Delivery of the therapeutic or imaging agent can occur through endocytosis of the liposomes. Such deliveries are known in the art. See, for example, Mastrobattista, et al., Immunoliposomes for the Targeted Delivery of Antitumor Drugs, Adv. Drug Del. Rev. (1999) 40:103-27.

In one embodiment, the attachment is by covalent means. In another embodiment, the attachment is by non-covalent means. For example, antibody targeting entities may be attached by a biotin-avidin biotinylated antibody sandwich, as shown in FIG. 14, to allow a variety of commercially available biotinylated antibodies to be used on the coated polymerized liposome. Specific vasculature targeting agents of use in the invention include (but are not limited to) anti-VCAM-1 antibodies (VCAM=vascular cell adhesion molecule); anti-ICAM-1 antibodies (ICAM=intercellular adhesion molecule); anti-integrin antibodies (e.g., antibodies directed against αᵥβ₃ integrins) such as LM609, described in International Patent Application WO 89/05155 and Cheresh et al. J. Biol. Chem. 262:17703-11 (1987), and Vitaxin, described in International Patent Application WO 9833919 and in Wu et al., Proc. Natl. Acad. Sci. USA 95(11):603742 (1998); and antibodies directed against P- and E-selectins, pleiotropin and endosialin, endoglin, VEGF receptors, PDGF receptors, EGF receptors, and prostate specific membrane antigen (PSMA).

In one embodiment of the invention, the vascular-targeted therapeutic agent is combined with an agent targeted directly towards tumor cells. This embodiment takes advantage of the fact that the neovasculature surrounding tumors is often highly permeable or “leaky,” allowing direct passage of materials from the bloodstream into the interstitial space surrounding the tumor. Alternatively, the vascular-targeted therapeutic agent itself can induce permeability in the tumor vasculature. For example, when the agent carries a radioactive therapeutic entity, upon binding to the vascular tissue and irradiating that tissue, cell death of the vascular epithelium will follow and the integrity of the vasculature will be compromised.

Accordingly, in one embodiment, the vascular-targeted therapeutic agent has two targeting entities: a targeting entity directed towards a vascular marker, and a targeting entity directed towards a tumor cell marker. In another embodiment, an antitumor agent is administered with the vascular-targeted therapy agent. The antitumor agent can be administered simultaneously with the vascular-targeted therapy agent, or subsequent to administration of the vascular-targeted therapy agent. In particular, when the vascular-targeted therapy agent is relied upon to compromise vascular integrity in the area of the tumor, administration of the antitumor agent is preferably done at the point of maximum damage to the tumor vasculature.

The antitumor agent can be a conventional antitumor therapy, such as cisplatin; antibodies directed against tumor markers, such as anti-Her2/neu antibodies (e.g., Herceptin); or tripartite agents, such as those described herein for vascular-targeted therapeutic agents, but targeted against the tumor cell rather than the vasculature. A summary of monoclonal antibodies directed against various tumor markers is given in Table I of U.S. Pat. No. 6,093,399, hereby incorporated by reference herein in its entirety. In general, when the vascular-targeted therapy agent compromises vascular integrity in the area of the tumor, the effectiveness of any drug which operates directly on the tumor cells can be enhanced.

The size of the vesicles can be adjusted for the particular intended end use including, for example, diagnostic and/or therapeutic use. As the size of the linking carrier can be manipulated readily, the overall size of the vascular-targeted therapeutic agents can be adapted for optimum passage of the particles through the permeable (“leaky”) vasculature at the site of pathology, as long as the agent retains sufficient size to maintain its desired properties (e.g., circulation lifetime, multivalency). Accordingly, the particles can be sized at 30, 50, 100, 150, 200, 250, 300 or 350 nm in size, as desired. In addition, the size of the particles can be chosen so as to permit a first administration of particles of a size that cannot pass through the permeable vasculature, followed by one or more additional administrations of particles of a size that can pass through the permeable vasculature. The size of the vesicles may preferably range from about 30 nanometers (nm) to about 400 nm in diameter, and all combinations and subcombinations of ranges therein. More preferably, the vesicles have diameters of from about 10 nm to about 500 nm, with diameters from about 40 nm to about 120 nm being even more preferred. In connection with particular uses, for example, intravascular use, including magnetic resonance imaging of the vasculature, it may be preferred that the vesicles be no larger than about 500 nm in diameter, with smaller vesicles being preferred, for example, vesicles of no larger than about 100 nm in diameter. It is contemplated that these smaller vesicles may perfuse small vascular channels, such as the microvasculature, while at the same time providing enough space or room within the vascular channel to permit red blood cells to slide past the vesicles.

While one major focus of the invention is the use of vascular-targeted therapy agent against the vasculature of tumors in order to treat cancer, the agents of the invention can be used in any disease where neovascularization or other aberrant vascular growth accompanies or contributes to pathology. Diseases associated with neovascular growth include, but are not limited to, solid tumors; blood born tumors such as leukemias; tumor metastasis; benign tumors, for example hemangiomas, acoustic neuromas, neurofibromas, trachomas, and pyogenic granulomas; rheumatoid arthritis; psoriasis; chronic inflammation; ocular angiogenic diseases, for example, diabetic retinopathy, retinopathy of prematurity, macular degeneration, corneal graft rejection, neovascular glaucoma, retrolental fibroplasia, rubeosis; arteriovenous malformations; ischemic limb angiogenesis; Osler-Webber Syndrome; myocardial angiogenesis; plaque neovascularization; telangiectasia; hemophiliac joints; angiofibroma; and wound granulation. Diseases of excessive or abnormal stimulation of endothelial cells include, but are not limited to, intestinal adhesions, atherosclerosis, restenosis, scleroderma, and hypertrophic scars, i.e., keloids.

Differing administration vehicles, dosages, and routes of administration can be determined for optimal administration of the agents; for example, injection near the site of a tumor may be preferable for treating solid tumors. Therapy of these disease states can also take advantage of the permeability of the neovasulature at the site of the pathology, as discussed above, in order to specifically deliver the vascular-targeted therapeutic agents to the interstitial space at the site of pathology.

Therapeutic Compositions

The present invention is also directed toward therapeutic compositions comprising the therapeutic agents of the present invention. Compositions of the present invention can also include other components such as a pharmaceutically acceptable excipient, an adjuvant, and/or a carrier. For example, compositions of the present invention can be formulated in an excipient that the animal to be treated can tolerate. Examples of such excipients include water, saline, Ringer's solution, dextrose solution, mannitol, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, Tris buffer, histidine, citrate, and glycine, or mixtures thereof, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, the composition can also include an immunopotentiator, such as an adjuvant or a carrier. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel.TM., Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark). Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

One embodiment of the present invention is a controlled release formulation that is capable of slowly releasing a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

Generally, the therapeutic agents used in the invention are administered to an animal in an effective amount. Generally, an effective amount is an amount effective to either (1) reduce the symptoms of the disease sought to be treated or (2) induce a pharmacological change relevant to treating the disease sought to be treated. For cancer, an effective amount includes an amount effective to: reduce the size of a tumor; slow the growth of a tumor; prevent or inhibit metastases; or increase the life expectancy of the affected animal.

Therapeutically effective amounts of the therapeutic agents can be any amount or doses sufficient to bring about the desired effect and depend, in part, on the condition, type and location of the cancer, the size and condition of the patient, as well as other factors readily known to those skilled in the art. The dosages can be given as a single dose, or as several doses, for example, divided over the course of several weeks.

The present invention is also directed toward methods of treatment utilizing the therapeutic compositions of the present invention. The method comprises administering the therapeutic agent to a subject in need of such administration.

The therapeutic agents of the instant invention can be administered by any suitable means, including, for example, parenteral, topical, oral or local administration, such as intradermally, by injection, or by aerosol. In the preferred embodiment of the invention, the agent is administered by injection. Such injection can be locally administered to any affected area. A therapeutic composition can be administered in a variety of unit dosage forms depending upon the method of administration. For example, unit dosage forms suitable for oral administration of an animal include powder, tablets, pills and capsules. Preferred delivery methods for a therapeutic composition of the present invention include intravenous administration and local administration by, for example, injection or topical administration. For particular modes of delivery, a therapeutic composition of the present invention can be formulated in an excipient of the present invention. A therapeutic reagent of the present invention can be administered to any animal, preferably to mammals, and more preferably to humans.

The particular mode of administration will depend on the condition to be treated. It is contemplated that administration of the agents of the present invention may be via any bodily fluid, or any target or any tissue accessible through a body fluid.

Preferred routes of administration of the cell-surface targeted therapeutic agents of the present invention are by intravenous, interperitoneal, or subcutaneous injection including administration to veins or the lymphatic system. While the primary focus of the invention is on vascular-targeted agents, in principle, a targeted agent can be designed to focus on markers present in other fluids, body tissues, and body cavities, e.g. synovial fluid, ocular fluid, or spinal fluid. Thus, for example, an agent can be administered to spinal fluid, where an antibody targets a site of pathology accessible from the spinal fluid. Intrathecal delivery, that is, administration into the cerebrospinal fluid bathing the spinal cord and brain, may be appropriate for example, in the case of a target residing in the choroid plexus endothelium of the cerebral spinal fluid (CSF)-blood barrier.

As an example of one treatment route of administration through a bodily fluid is one in which the disease to be treated is rheumatoid arthritis. In this embodiment of the invention, the invention provides therapeutic agents to treat inflamed synovia of people afflicted with rheumatoid arthritis. This type of therapeutic agent is a radiation synovectomy agent. Individuals with rheumatoid arthritis experience destruction of the diarthroidal or synovial joints, which causes substantial pain and physical disability. The disease will involve the hands (metacarpophalangeal joints), elbows, wrists, ankles and shoulders for most of these patients, and over half will have affected knee joints. Untreated, the joint linings become increasingly inflamed resulting in pain, loss of motion and destruction of articular cartilage. Chemicals, surgery, and radiation have been used to attack and destroy or remove the inflamed synovium, all with drawbacks.

The concentration of the radiation synovectomy agent varies with the particular use, but a sufficient amount is present to provide satisfactory radiation synovectomy. For example, in radiation synovectomy of the hip, the concentration of the agent will generally be higher than when used for the radiation synovectomy of the wrist joints. The radiation synovectomy composition is administered so that preferably it remains substantially in the joint for 20 half-lifes of the isotope although shorter residence times are acceptable as long as the leakage of the radionuclide is small and the leaked radionuclide is rapidly cleared from the body.

The radiation synovectomy compositions may be used in the usual way for such procedures. For example, in the case of the treatment of a knee-joint, a sufficient amount of the radiation synovectomy composition to provide adequate radiation synovectomy is injected into the knee-joint. There are a number of different techniques which can be used and the appropriate technique varies on the joint being treated. An example for the knee joint can be found, for example, in Nuclear Medicine Therapy, J. C. Harbert, J. S. Robertson and K. D. Reid, 1987, Thieme Medical Publishers, pages 172-3.

The route of administration through the synovia may also be useful in the treatment of osteoarthritis. Osteoarthritis is a disease where cartilage degradation leads to severe pain and inability to use the affected joint. Although age is the single most powerful risk factor, major trauma and repetitive joint use are additional risk factors. Major features of the disease include thinning of the joint, softening of the cartilage, cartilage ulcers, and abraded bone. Delivery of agents by injection of targeted carriers to synovial fluid to reduce inflammation, inhibit degradative enzymes, and decrease pain are envisioned in this embodiment of the invention.

Another route of administration is through ocular fluid. In the eye, the retina is a thin layer of light-sensitive tissue that lines the inside wall of the back of the eye. When light enters the eye, it is focused by the cornea and the lens onto the retina. The retina then transforms the light images into electrical impulses that are sent to the brain through the optic nerve.

The macula is a very small area of the retina responsible for central vision and color vision. The macula allows us to read, drive, and perform detailed work. Surrounding the macula is the peripheral retina which is responsible for side vision and night vision. Macular degeneration is damage or breakdown of the macula, underlying tissue, or adjacent tissue. Macular degeneration is the leading cause of decreased visual acuity and impairment of reading and fine “close-up” vision. Age-related macular degeneration (ARMD) is the most common cause of legal blindness in the elderly.

The most common form of macular degeneration is called “dry” or involutional macular degeneration and results from the thinning of vascular and other structural or nutritional tissues underlying the retina in the macular region. A more severe form is termed “wet” or exudative macular degeneration. In this form, blood vessels in the choroidal layer (a layer underneath the retina and providing nourishment to the retina) break through a thin protective layer between the two tissues. These blood vessels may grow abnormally directly beneath the retina in a rapid uncontrolled fashion, resulting in oozing, bleeding, or eventually scar tissue formation in the macula which leads to severe loss of central vision. This process is termed choroidal neovascularization (CNV).

CNV is a condition that has a poor prognosis; effective treatment using thermal laser photocoagulation relies upon lesion detection and resultant mapping of the borders. Angiography is used to detect leakage from the offending vessels but often CNV is larger than indicated by conventional angiograms since the vessels are large, have an ill-defined bed, protrude below into the retina and can associate with pigmented epithelium.

Neovascularization results in visual loss in other eye diseases including neovascular glaucoma, ocular histoplasmosis syndrome, myopia, diabetes, pterygium, and infectious and inflammatory diseases. In histoplasmosis syndrome, a series of events occur in the choroidal layer of the inside lining of the back of the eye resulting in localized inflammation of the choroid and consequent scarring with loss of function of the involved retina and production of a blind spot (scotoma). In some cases, the choroid layer is provoked to produce new blood vessels that are much more fragile than normal blood vessels. They have a tendency to bleed with additional scarring, and loss of function of the overlying retina. Diabetic retinopathy involves retinal rather than choroidal blood vessels resulting in hemorrhages, vascular irregularities, and whitish exudates. Retinal neovascularization may occur in the most severe forms. When the vasculature of the eye is targeted, it should be appreciated that targets may be present on either side of the vasculature.

Delivery of the agents of the present invention to the tissues of the eye can be in many forms, including intravenous, ophthalmic, and topical. For ophthalmic topical administration, the agents of the present invention can be prepared in the form of aqueous eye drops such as aqueous suspended eye drops, viscous eye drops, gel, aqueous solution, emulsion, ointment, and the like. Additives suitable for the preparation of such formulations are known to those skilled in the art. In the case of a sustained-release delivery system for the eye, the sustained-release delivery system may be placed under the eyelid or injected into the conjunctiva, sclera, retina, optic nerve sheath, or in an intraocular or intraorbitol location. Intravitreal delivery of agents to the eye is also contemplated. Such intravitreal delivery methods are known to those of skill in the art. The delivery may include delivery via a device, such as that described in U.S. Pat. No. 6,251,090 to Avery.

In a further embodiment, the therapeutic agents of the present invention are useful for gene therapy. As used herein, the phrase “gene therapy” refers to the transfer of genetic material (e.g., DNA or RNA) of interest into a host to treat or prevent a genetic or acquired disease or condition. The genetic material of interest encodes a product (e.g., a protein polypeptide, peptide or functional RNA) whose production in vivo is desired. For example, the genetic material of interest can encode a hormone, receptor, enzyme or polypeptide of therapeutic value. In a specific embodiment, the subject invention utilizes a class of lipid molecules for use in non-viral gene therapy which can complex with nucleic acids as described in Hughes, et al., U.S. Pat. No. 6,169,078, incorporated by reference herein in its entirety, in which a disulfide linker is provided between a polar head group and a lipophilic tail group of a lipid.

These therapeutic compounds of the present invention effectively complex with DNA and facilitate the transfer of DNA through a cell membrane into the intracellular space of a cell to be transformed with heterologous DNA. Furthermore, these lipid molecules facilitate the release of heterologous DNA in the cell cytoplasm thereby increasing gene transfection during gene therapy in a human or animal.

Cationic lipid-polyanionic macromolecule aggregates may be formed by a variety of methods known in the art. Representative methods are disclosed by Felgner et al., supra; Eppstein et al. supra; Behr et al. supra; Bangham, A. et al. M. Mol. Biol. 23:238, 1965; Olson, F. et al. Biochim. Biophys. Acta 557:9, 1979; Szoka, F. et: al. Proc. Natl. Acad. Sci. 75: 4194, 1978; Mayhew, E. et al. Biochim. Biophys. Acta 775:169, 1984; Kim, S. et al. Biochim. Biophys. Acta 728:339, 1983; and Fukunaga, M. et al. Endocrinol. 115:757, 1984. In general aggregates may be formed by preparing lipid particles consisting of either (1) a cationic lipid or (2) a cationic lipid mixed with a colipid, followed by adding a polyanionic macromolecule to the lipid particles at about room temperature (about 18 to 26° C.). In general, conditions are chosen that are not conducive to deprotection of protected groups. In one embodiment, the mixture is then allowed to form an aggregate over a period of about 10 minutes to about 20 hours, with about 15 to 60 minutes most conveniently used. Other time periods may be appropriate for specific lipid types. The complexes may be formed over a longer period, but additional enhancement of transfection efficiency will not usually be gained by a longer period of complexing.

The compounds and methods of the subject invention can be used to intracellularly deliver a desired molecule, such as, for example, a polynucleotide, to a target cell. The desired polynucleotide can be composed of DNA or RNA or analogs thereof. The desired polynucleotides delivered using the present invention can be composed of nucleotide sequences that provide different functions or activities, such as nucleotides that have a regulatory function, e.g., promoter sequences, or that encode a polypeptide. The desired polynucleotide can also provide nucleotide sequences that are antisense to other nucleotide sequences in the cell. For example, the desired polynucleotide when transcribed in the cell can provide a polynucleotide that has a sequence that is antisense to other nucleotide sequences in the cell. The antisense sequences can hybridize to the sense strand sequences in the cell. Polynucleotides that provide antisense sequences can be readily prepared by the ordinarily skilled artisan. The desired polynucleotide delivered into the cell can also comprise a nucleotide sequence that is capable of forming a triplex complex with double-stranded DNA in the cell.

Imaging

The present invention is directed to imaging agents displaying important properties in medical diagnosis. More particularly, the present invention is directed to magnetic resonance imaging contrast agents, such as gadolinium, ultrasound imaging agents, or nuclear imaging agents, such as Tc-99m, In-111, Ga-67, Rh-105, I-123, Nd-147, Pm-151, Sm-153, Gd-159, Tb-161, Er-171, Re-186, Re-188, and Tl-201.

This invention also provides a method of diagnosing abnormal pathology in vivo comprising, introducing a plurality of targeting image enhancing polymerized particles targeted to a molecule involved in the abnormal pathology into a bodily fluid contacting the abnormal pathology, the targeting image enhancing polymerized particles attaching to a molecule involved in the abnormal pathology, and imaging in vivo the targeting image enhancing polymerized particles attached to molecules involved in the abnormal pathology.

Diagnostics

The present invention further provides methods and reagents for diagnostic purposes. Diagnostic assays contemplated by the present invention include, but are not limited to, receptor-binding assays, antibody assays, immunohistochemical assays, flow cytometry assays, genomics and nucleic acid detection assays. High-throughput screening arrays and assays are also contemplated.

This invention provides various methods for in vitro assays. For example, antibody-conjugated polymerized liposomes, according to this invention, provide an ultra-sensitive diagnostic assay for specific antigens in solution. Polymerized liposomes of this invention having a chelator head group chelated to spectroscopically distinct ions provide high sensitivity for immunoassays as well as ligand and receptor-based assays. Polymerized liposomes of this invention having a fluorophore head group provide a method for detection of glycoproteins on cell surfaces.

Liposomes useful in diagnostic assays are described in U.S. Pat. No. 6,090,408, entitled “Use of Polymerized Lipid Diagnostic Agents,” and U.S. Pat. No. 6,132,764, entitled “Targeted Polymerized Liposome Diagnostic and Treatment Agents,” each incorporated by reference herein in its entirety.

In one embodiment of this invention, a targeting polymerized liposome particle comprises: an assembly of a plurality of liposome forming lipids each having an active hydrophilic head group linked by a bifunctional linker portion to the liposome forming lipid, and a hydrophobic tail group having a polymerizable functional group polymerized with a polymerizable functional group of an adjacent hydrophobic tail group of one of the plurality of liposome forming lipids, at least a portion of the hydrophilic head groups having an attached targeting active agent for attachment to a specific biological molecule. In another embodiment, the targeting polymerized liposome particle has a second portion of the hydrophilic head groups with functional surface groups attached to an image contrast enhancement agent to form a targeting image enhancing polymerized liposome particle. In yet another embodiment, a portion of the hydrophilic head groups have functional surface groups attached to or encapsulating a treatment agent for interaction with a biological site at or near the specific biological molecule to which the particle attaches, forming a targeting delivery polymerized liposome particle or a targeting image enhancing delivery polymerized liposome particle.

This invention provides a method of assaying abnormal pathology in vitro comprising, introducing a plurality of liposomes of the present invention to a molecule involved in the abnormal pathology into a fluid contacting the abnormal pathology, the targeting polymerized liposome particles attaching to a molecule involved in the abnormal pathology, and detecting in vitro the targeting polymerized liposome particles attached to molecules involved in the abnormal pathology.

Exemplary Lipid Constructs and Uses

Polymerized Vesicles

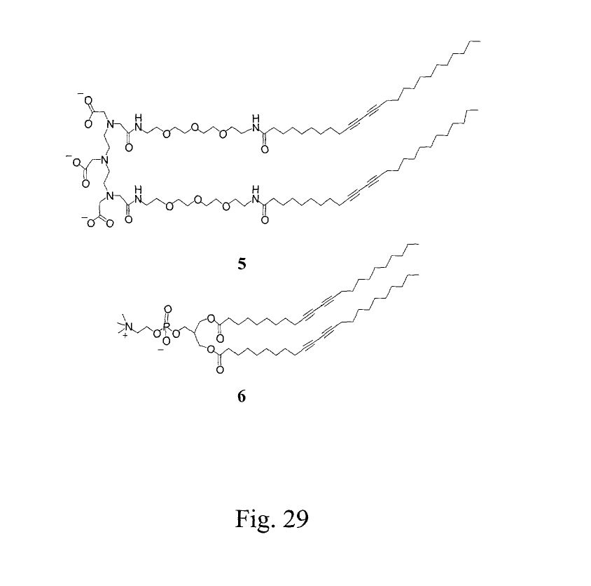

Chelating polymerized vesicles (CPVs), prepared as described in Example 14, consist of diacetylene containing lipids 1,2-bis(10,12-tricosadiynoyl)-sn-glycero-3-phosphocholine (BisT-PC, 6) (FIG. 29) and 1-5 mole percent of the diethylenetriaminetriacetic acid (DTTA) lipid derivative (5) (FIG. 29) by extrusion and polymerization with UV light to generate particles with mean diameters of 60-80 nm as determined by dynamic light scattering. Diacetylenic lipids cross-link during exposure to UV light resulting in a highly conjugated backbone consisting of alternating double and triple carbon-carbon bonds (D. S. Johnston, S. Sanghera, M. Pons, D. Chapman, Biochim Biophys Acta 602, 57-69. (1980)).

Attachment of Peptides and Antibodies to Vesicles

Peptide GRGDS, murine antibody LM609 (P. C. Brooks, et al., J Clin Invest 96, 1815-22 (1995)), or the humanized antibody Vitaxin (H. Wu, et al., Proc Natl Acad Sci U S A 95, 6037-42 (1998)), all of which bind the human αᵥβ₃ integrin, are attached to the surface carboxyl groups of the polymerized vesicles using EDAC chemistry as described in Examples 20 and 21, which results primarily in amide bond formation with nucleophilic groups such as the amines on N-terminus amino groups or lysines that are present on the protein or peptide (G. T. Hermanson, Bioconjugate Techniques (Academic Press, San Diego, 1996)). Other antibodies attached to CPVs include LM609, a murine anti- human αᵥβ₃ integrin antibody (Brooks, 1995, ibid.), and rat antibodies with specificity to mouse endothelial proteins including the αᵥ integrin subunit, and the VEGF receptor 2, also known as KDR or Flk-1. The resulting 75-150 nm conjugates were purified by size exclusion chromatography with baseline resolution of the conjugates from unbound antibodies or peptides.

The presence of antibody on purified antibody-CPV conjugates was confirmed by sandwich ELISA as described in Example 20, using an anti-human IgG antibody to capture the antibody-CPV conjugate, and an HRP-anti human IgG antibody conjugate to detect the antibody. Further purification of the conjugates by size exclusion chromatography using elution buffers containing 150 mM sodium chloride shows that the coupling is covalent, since non-covalently bound antibodies do not adhere to the vesicles under these conditions.

Targeting of Vitaxin-CPVs and the GRGDS peptide-CPVs was further demonstrated by inhibition of αᵥβ₃ integrin-mediated binding of M21 human melanoma cells to fibrinogen and in a binding assay with purified αᵥβ₃ integrin as described in Example 22. Vitaxin- and GRGDS peptide-CPVs labeled with yttrium-90 bind to purified integrin-coated 96-well plates in a concentration dependent manner (FIG. X). This assay generates signal only if the targeting antibody or peptide and the yttrium 90 are bound to the same vesicle. Vitaxin-CPVs inhibit the adhesion of M21 cells to fibrinogen with an IC50 of 11 μg/mL, which corresponds to 0.7 nm Vitaxin. The IC50 for Vitaxin is 2 nm, and CPVs without antibody do not inhibit the adhesion of M21 cells to fibrinogen.

Attachment of Trivalent Metals to the Vesicles

Naturally occurring yttrium-89 as well as isotopes yttrium-90, and indium-111 are attached to the polymerized vesicles or liposomes via chelation to the triacetic acid DTTA head group of lipid 5 as described in Example 15. The labeling efficiency is greater than 98% with a binding capacity for yttrium-90 of approximately 10 mCi per mg of lipid. The metal binding capacities of CPVs and Vitaxin-CPVs are indistinguishable, thus the use of EDAC does not significantly alter the concentration of chelating DTTA groups under the conditions used to attach antibodies and peptides. The effect of pH on yttrium-90 binding efficiency was examined in acetate, MES, and HEPES buffers and is pH independent from pH 5-7. CPVs may also be labeled with indium-111, a gamma-emitting isotope commonly used for in-vivo imaging studies. The labeling efficiencies were 90-98% at loading levels of 50-500 μCi per mg of CPV. Because of the high metal binding capacity, CPVs also bind yttrium-90 and indium-111 simultaneously. Sequential loading experiments with 0.1 or 1 mCi of each isotope per mg of CPV resulted in 95-99% binding of both isotopes.

Specific labeling of the DTTA chelator on the vesicles was demonstrated by incubation of the CPV-⁹⁰Y complexes with the weak chelator citrate, and the strong chelator diethylaminetriaminepentaacetic acid (DTPA) at DTTA-lipid concentrations of 0.56-560 μM. The metal complexes are stable in the presence of 500 mM citrate and about 90% of the yttrium is retained in the presence of 1 mM DTPA following a 30-minute incubation of the vesicle-⁹⁰Y complex. Polymerized vesicles prepared solely from BisT-PC or those containing both BisT-PC and 5-30 mole percent of a succinylated phosphatidylethanolamine head group as the sole source of carboxyl functionality do not bind yttrium-90 efficiently in the presence of citrate. These results suggests that coordination of yttrium 90 by the triacetic acid head group is required for the formation of a stable vesicle-yttrium complex.

The concentration of the DTTA head group in CPV solutions does not appear to be altered significantly when presented on the surfaces of the vesicles. This conclusion may be drawn from the stability of the CPV-90Y complexes in the presence of a 2-2000 fold excess DTPA, and also from titrations of the chelating-lipid that show that the measured concentration of chelator matches the calculated concentration. The experiments were performed as described in Examples 18 and 19. These titration experiments were performed by adding “cold” yttrium-89 to CPVs followed by both the addition of the yttrium-90 isotope, and measurement of the yttrium-90 bound to vesicles. As the amount of yttrium-89 increases, the binding of yttrium-90 decreases due to saturation of the binding sites on the CPVs which results in inhibition of yttrium-90 binding. The concentration of yttrium-89 at which yttrium-90 no longer binds is equal to the concentration of chelation sites. Alternatively, the titrations were performed by the addition of tracer amounts of yttrium-90 to yttrium-89, and adding this mixture, which contains excess yttrium-89, to vesicles. Measured concentrations of the DTTA head group present in solution are in agreement with calculated concentrations. For CPVs containing 1 and 5 mole percent of the DTTA-lipid 2, the calculated concentrations of 0.11 and 0.55 mM agree closely with the measured concentrations of 0.5 and 0.1 mM of the DTTA chelator.

In-vitro Targeting of Integrin-targeted Vesicles

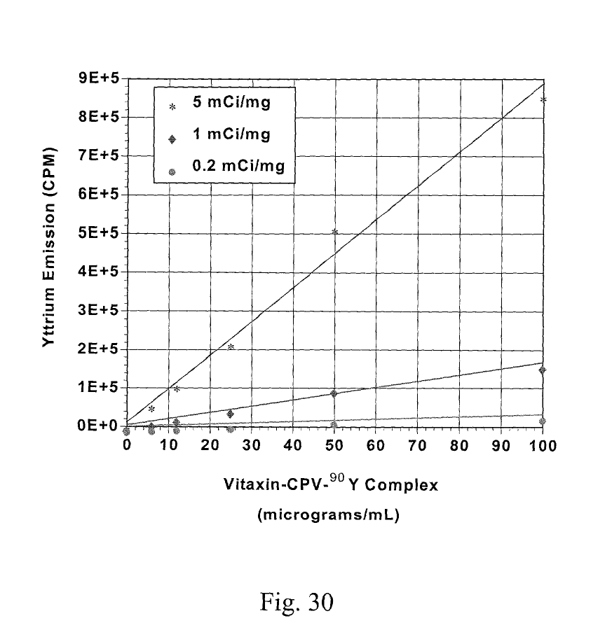

Vitaxin-CPV and RGD peptide-CPV conjugates, which also bind yttrium-90 with high efficiency, target the αᵥβ₃ integrin in-vitro in a radiometric binding assay performed as described in Example 21. In a typical assay, Vitaxin-CPV conjugates are labeled with 0.1-5 mCi of yttrium-90 per milligram of CPV conjugate, and this solution is diluted serially to 6, 12, 25, and 50 μg/mL. Incubation of the Vitaxin-CPV-⁹⁰Y complex with human αᵥβ₃ integrin on 96-well plates results in a linear response in signal as a function of concentration with signal to background ratios of up to 270 to 1. Additionally, the amount of yttrium-90 added to the CPV solutions directly correlates with differences in the observed signals in this assay. For yttrium-90 loadings of 0.2, 1 and 5 mCi, which differ by factors of 5, the corresponding signals obtained in the binding assay differed by factors of 5.0±0.3 for 6-60 μg/mL of the Vitaxin-CPV-⁹⁰Y complex. These results, shown in graphical form in FIG. 30, demonstrate that yttrium-90 binding is controllable in-vitro, and thus the dose delivered by a targeted-CPV in-vivo may be controlled to optimize efficacy and toxicity.

Stability of Antibody-CPV-isotope Conjugates in-vitro

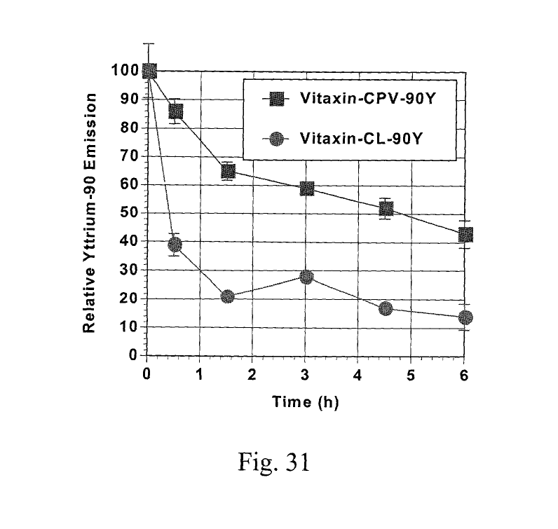

In order to assess the stability of conjugates in serum, the Vitaxin-CPV-⁹⁰Y complex containing 5 mole percent chelator 5 and BisT-PC 6 was incubated in rabbit serum at 37° C. and compared to Vitaxin-PC/cholesterol chelating liposomes containing chelator 5, cholesterol, and egg phosphocholine (Vitaxin-CL-⁹⁰Y complexes) at molar ratios of 5/28/67 using the radiometric αᵥβ₃ integrin binding assay. Vitaxin-CPV conjugates were significantly more stable than Vitaxin-CL-⁹⁰Y complexes (FIG. 31). Vitaxin-CPV-⁹⁰Y conjugates have a half-life in serum of approximately 4.8 hours compared to approximately 0.4 hours for Vitaxin-PC/cholesterol liposomes. Vitaxin-liposome-⁹⁰Y conjugates containing lipids 5 and 6 that were not polymerized were not stable in serum and gave 5-fold lower signals than the corresponding polymerized vesicles, as shown in FIG. 31.

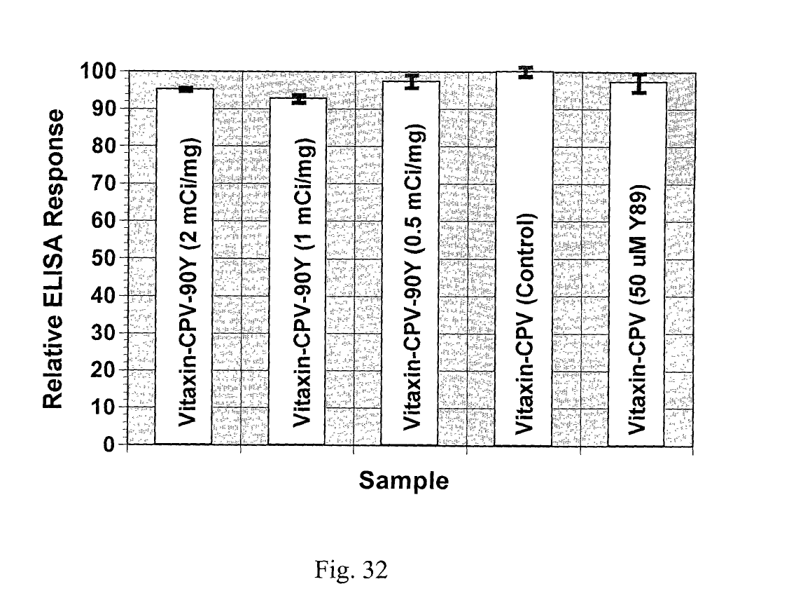

Yttrium-90 emission does not affect the immunoreactivity of the Vitaxin-CPV conjugates. Radiolysis, which is the loss of immunoreactivity of radiolabeled conjugates during exposure to radioisotopes, was examined by labeling Vitaxin-CPVs at 0.5, 1, and 2 mCi of yttrium-90 per mg of Vitaxin-CPV conjugate. The corresponding loading levels calculated per milligram of antibody are approximately 20, 40, and 80 mCi of yttrium-90 per milligram of Vitaxin. After storage of the Vitaxin-CPV-⁹⁰Y conjugates at 1 mg/mL in 50 mM histidine buffer containing 5 mM citrate at pH 7.4 at 4° C. for 60 days, the conjugates were analyzed by ELISA with the αᵥβ₃ integrin, and compared to controls without yttrium or with naturally occurring yttrium-89 at 50 μM. All complexes retained 93-97% of the ELISA signal of the Vitaxin-CPV without yttrium. A complex that was labeled with 50 μM yttrium-89 retained 97% of the ELISA response relative to the control without yttrium. These results, shown in FIG. 32, indicate that yttrium does not significantly affect the immunoreactivity of Vitaxin-CPV conjugates.

Imaging of the Vx2 Carcinoma in Rabbits

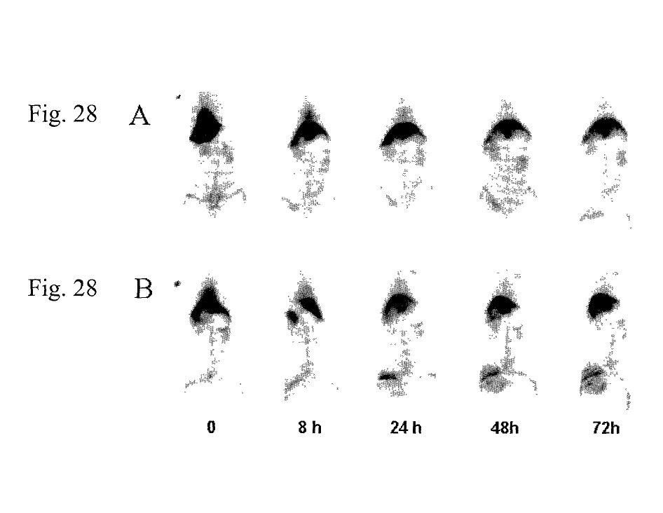

The accumulation of CPVs targeted to the αᵥβ₃ integrin was reproducibly demonstrated in the Vx2 rabbit carcinoma model. For these studies, CPV conjugates were labeled with indium-111, a gamma emitting isotope with a half-life of 67 h, and administered to rabbits bearing Vx2 tumors of similar size in the thighs. Serial images acquired immediately after injection and at 8, 24, 48, and 72 hours show significant accumulation in the tumor with 22% of the total body counts located in the tumor at 72 h (FIG. 28B) compared to approximately 3% for the untargeted vesicle (FIG. 28A). The other significant site of accumulation of indium-111 was in the liver.

Targeted nanoscale radioconjugates for the delivery of the beta-emitting isotope yttrium-90 and other isotopes are novel and promising agents. These conjugates are constructed from metal chelating polymerized vesicles (CPVs) containing a diethylenetriaminetriacetic acid (DTTA) head group. Because CPVs contain a high molar percentage of this head group, the carboxyl groups of DTTA may be used for both the conjugation of targeting agents and the binding of metal ions. Conjugation using the water-soluble carbodiimide EDAC to activate the surface carboxyl groups does not have a significant effect on yttrium binding since the metal binding capacity of both CPVs and Vitaxin-CPVs are indistinguishable. The attachment of yttrium to the targeted CPVs is achieved by addition of the isotope to the CPV at room temperature and is greater than 98% efficient. Therefore, purification of unbound isotope from the CPV-⁹⁰Y complexes is not required. These agents may also be labeled simultaneously with indium-111 potentially allowing for the monitoring delivery to the target site.

CPVs have a high capacity for metal ion binding. Particles ranging in size from 60-150 nm contain approximately 1600-9000 DTTA-lipid molecules for particles containing 5 mole percent of this lipid, based on surface area calculations assuming that the surface area for the DTTA head group is similar to the 65 Å² reported for 1,2-distearoylphosphatidylcholine (P. Balgavy, et al., Biochim Biophys Acta 1512, 40-52. (2001)). The antibody-CPV conjugates prepared at 25 μg of antibody per milligram of vesicle contain an average of approximately 2-5 antibodies per vesicle after accounting for reaction yields of 40-90%.

Therapies targeting macromolecules to proteins up-regulated on endothelial cells in tumor vasculature are advantageous because the target is easily accessible whereas targeting tumor cells is difficult because of low diffusion rates due to high interstitial pressure in solid tumors (R. K. Jain, Adv Drug Deliv Rev 46, 149-68. (2001)). In order to target macromolecules to endothelial markers, we prepared vesicles targeting the αᵥβ₃ integrin using the integrin binding peptide GRGDS or the humanized antibody Vitaxin. Thus, in-vitro-targeting may be achieved by the attachment of both small molecules and different antibodies to CPVs.

The immunoreactivity of Vitaxin-CPVs may have been affected modestly relative to Vitaxin in an ELISA with purified αᵥβ₃ integrin. Vitaxin-CPV conjugates give 2-6 fold lower signals relative to Vitaxin at identical antibody concentrations. However, this assay does not measure affinity, and the reduction in signals may be a result of modest changes in binding kinetics or impaired binding of either one or both of the binding elements in this assay, namely the integrin recognition site of the antibody, and the Fc region of the antibody.

Vitaxin- or GRGDS-CPVs target the αᵥβ₃ integrin in-vitro. In addition to binding to purified αᵥβ₃ integrin, these conjugates inhibit the binding of fibronectin to purified αᵥβ₃ integrin as well as the binding of M21 melanoma cells to fibrinogen. These results show that the integrin-targeted CPVs inhibit the binding of this receptor to its natural substrates, and that these conjugates recognize both purified integrin and cellular integrin.

Vitaxin- or GRGDS-CPVs labeled with yttrium-90 also bind the integrin target in-vitro. Binding to purified αᵥβ₃ integrin was achieved in both buffered solutions and in the presence of both rabbit and human serum, which demonstrates potential for targeting in-vivo since serum does not significantly interfere with binding to the target in-vitro. Vitaxin-CPVs labeled with 0.2, 1, and 5 mCi of ⁹⁰Y per mg of vesicle give the expected increases in signal in a radiometric binding assay to purified αᵥβ₃ integrin, demonstrating that yttrium-90 binding is controllable in-vitro. Thus, the dose delivered by a targeted-CPV in-vivo may be controlled to optimize efficacy and toxicity.

CPVs are stable in the presence of yttrium-90 and in the presence of serum. Vitaxin-CPV-⁹⁰Y complexes do not show significant loss of immunoreactivity as a result of radiolysis at loading levels of 0.5-2 mCi per mg of lipid, which corresponds to 20-80 mCi per mg of antibody. In contrast, the immunoreactivity for an antibody-⁹⁰Y complex has been reported to decrease by 72% at loading levels of 4 mCi per mg of antibody over a 72 hour period (Q. A. Salako, R. T. O'Donnell, S. J. DeNardo, J Nucl Med 39, 667-70. (1998)). In rabbit serum, both Vitaxin- and GRGDS-CPV-⁹⁰Y complexes have a half-life of approximately 260 minutes, which is about 10-fold higher than that of a Vitaxin-liposome conjugate consisting of Vitaxin and a steroyl-based phosphatidylcholine, cholesterol, and DTTA-chelator 1,2-dimyristoyl-sn-glycero-3-phosphoethanolamidotriamine tetraacetic acid. A similar vesicle prepared using chelator 5 also showed poor stability under identical conditions. This stability is related to the stability of the vesicle, the Vitaxin-vesicle complex, and the vesicle-⁹⁰Y complex. Examination of the stability of the vesicle-⁹⁰Y complex in rabbit serum by size exclusion chromatography showed that the signal losses were primarily the result of dissociation of the yttrium from the vesicle. Dissociation of yttrium from the complex is not likely related to vesicle instability since vesicles prepared containing a phosphatidylethanolamine lissamine rhodamine B lipid remain intact in serum under identical conditions. This conclusion is further supported by studies with ¹⁴C labeled lipids that show that the vesicles remain intact in serum (Q. F. Ahkong, C. Tilcock, Int J Rad Appl Instrum B 19, 831-40. (1992)).

### EXAMPLES

### Example 1

### Synthesis of a Differentially-protected Branched Polylysine Macromolecular Linking Carrier

Lysine t-butyl ester is readily synthesized from commercially available lysine (Calbiochem-Novabiochem Corp., San Diego, Calif.) and isobutylene using the procedure described in Bodanszky and Bodanszky, The Practice of Peptide Synthesis, New York: Springer-Verlag, 1984, pp. 48-49. N-α-Fmoc-N-ε-Fmoc-lysine O-Pfp ester (N-α,ε,-di-Fmoc-L-lysine pentafluorophenyl ester, Calbiochem-Novabiochem Corp., San Diego, Calif.) is reacted with lysine t-butyl ester to form N-α-(N′-α-Fmoc-N′-ε-Fmoc-lysyl)-N-ε-(N″-α-Fmoc-N″-ε-Fmoc-lysyl)lysine t-butyl ester. If additional branching is desired, the Fmoc groups are removed with piperidine and the resulting deprotected amines are again reacted with N-α-Fmoc-N-ε-Fmoc-lysine O-Pfp ester; the process is reputed until the desired level of branching from the amino groups of the lysine moiety is reached.

Branching at the carboxyl group is readily accomplished by using N-α-Fmoc-glutamic acid α-, γ-t-butyl ester or N-α-Fmoc-aspartic acid α-, β-t-butyl ester. The di-t-butyl esters are readily prepared from Fmoc-Glu(OtBu)-OH or Fmoc-Asp(OtBu)-OH (Calbiochem-Novabiochem) and isobutylene using the method for esterifying lysine, above. The Fmoc group is then removed from the amino acid to yield (for the glutamate derivative) glutamic acid α-, γ-t-butyl ester. The t-butyl group of the branched lysine is removed using 95% trifluoroacetic acid. The amino group of glutamic acid α-, γ-t-butyl ester is condensed with the free carboxylic acid of the branched lysine using diisopropylcarbodiimide and 1-hydroxybenzotriazole activation chemistry. The cycle of 95% TFA deprotection and coupling can be repeated should additional branching at the carboxyl groups be desired.

The resulting branched lysine/glutamate macromolecule contains Fmoc-protected amino groups which can be selectively deprotected with piperidine, and t-butyl protected carboxyl groups which can be selectively deprotected with 95% trifluoroacetic acid. These differentially-protected groups can be used to attach therapeutic entities at one specific location on the molecule and targeting entities at another specific location.

### Example 2

### Synthesis of Poly(Glu-Lys) Polymer

Another polypeptide polymer suitable for use as a linking carrier is poly(glutamic acid-lysine) (poly(glutamyl-lysine) or poly(EK)). N-α-Fmoc glutamic acid y-benzyl ester (Fmoc-Glu(OBzl)-OH) is coupled to N-E-CBZ lysine t-butyl ester (H-Lys(Z)-tBu) (both reagents are commercially available from Calbiochem-Novabiochem, San Diego, Calif.) using diisopropylcarbodiimide and 1-hydroxybenzotriazole. The resulting dipeptide, Fmoc-Glu(OBzl)-Lys(Z)-tBu, can be deprotected using piperidine followed by 95% trifluoroacetic acid to yield H-Glu(OBzl)-Lys(Z)-OH. The dipeptide unit can then be freely polymerized to form a mixture of varying chain lengths, by carbodiimide or other condensation. Alternatively, if a defined length is desired, deprotection of the amino terminal with piperidine to afford H-Glu(OBzl)-Lys(Z)-OtBu and deprotection of the carboxyl terminal with 95% trifluoroacetic acid to afford Fmoc-Glu(OBzl)-Lys(Z)-OH enables condensation of the two dipeptides with carbodiimides to give Fmoc-Glu(OBzl)-Lys(Z)-Glu(OBzl)-Lys(Z)-OtBu. Repetition of this cycle can give poly(Glu(OBzl)-Lys(Z)) of a defined length. For either the random polymer or the defined-length polymer, the benzyl protecting group on glutamic acid and the CBZ protecting group on lysine can be removed simultaneously using either H₂/Pd or strong acids such as liquid HF or trifluoromethanesulfonic acid. This makes available both free amino and free carboxyl groups for use in attaching targeting and therapeutic moieties. The free amino groups can be reprotected with Boc, Bpoc or Fmoc groups in order to prevent reaction during derivatization of the carboxylate groups, by using standard methods in the field of peptide chemistry.

### Example 3

### Preparation of Chelator Lipid and Polymerized Liposomes I

Polymerizable lipids having Gd⁺³ and PDA headgroups were synthesized by first preparing the succinimidyl ester by stirring pentacosadiynoic acid (PDA, Lancaster; 10.0 g, 26.7 mmol), N-hydroxysuccinimide (NHS, Aldrich; 5.00 g, 43.4 mmol) and 1-(3-dimethylaminopropyl)-3-ethylcarbodiimide hydrochloride (EDAC, Aldrich; 6.01 g, 31.3 mmol) in 660 ml CH₂Cl₂ at room temperature and shielded from light. The reaction was followed by thin layer chromatography (CHCl₃/MeOH, 8/1) and deemed complete after approximately 5 hours. The solution was washed with water, 1% HCl, saturated sodium bicarbonate and brine. The organic phase was then dried with MgSO₄, filtered, and concentrated under reduced pressure to yield the N-succinimidyl-10,12-pentacosadiynoic acid ester as a slightly yellow solid (10.84 g; 23.0 mmol; 86%).

The succinimidyl ester was dissolved in CH₂Cl₂ (250 ml) and then slowly added, in dropwise fashion, to a stirred solution of 1,11-diamino-3,6,9-trioxyundecane (9.13 g, 61.6 mmol; Texaco) in CH₂Cl₂, (110 ml) over a 16 hour period at room temperature and shielded from light. The resulting solution was concentrated to a thick slurry and chromatographed on silica gel using a gradient of CHCl₃/MeOH (1/0 to 8/1). The homogeneous fractions were pooled and evaporated under reduced pressure to result in the desired lipid, (1′-N-11′-amino-3′,6′-dioxyundecanoyl)-10,12-pentacosadiynamide, as a white solid (4.40 g; 38.1%). This product must be handled with care as it spontaneously polymerizes in the solid state when it is pure. It is more stable in solution at 4° C., but should be used as soon as possible after preparation.

The above-prepared aminoamide (4.40 g; 8.78 mmol) and DTPA (1.56 g; 4.37 mmol) were stirred in pyridine (25 ml) overnight, shielded from the light. The solvent was evaporated and the residue coevaporated with methanol to dryness twice to result in an oil free from pyridine. The residue was dissolved in acetone and the product allowed to precipitate from solution after overnight storage at 4° C. Filtration resulted in the desired chelator lipid, bis-N-[2-ethyl-N-′carboxymethyl, N′-carboxymethyl (1′-N-′″,11′-N″″-3′,6′-dioxyundecanoyl)amide-1″,12″-pentacosadiynamide]-glycine, as a white amorphous powder (3.30 g; 55%). Further purification can be achieved by crystallization from methanol (40 mg/ml; m.p. 128.5-129.5° C. (decomp.).

The chelator lipid, as prepared above, was heated with GdCl₃.6H₂O or DyCl₃.6H₂O (0.95-0.98 equiv.) in methanol. The solvent was evaporated and the residue coevaporated with methanol to remove all traces of generated HCl. The resulting lanthanide chelate lipids, bis-N-[2-ethyl-N-′carboxymethyl,N′-carboxymethyl (1′-N-′″,11′-N″″-3′,6′dioxyundecanoyl)amide-1″,12″-pentacosadiynamide]-glycine-lanthanide, gadolinium or dysprosium complexes, were then stored as methanolic solutions at 4° C., shielded from light. The identity of the synthesized chelates was confirmed by FAB-MS.

Paramagnetic polymerized lipids were formed by mixing a 1:9 molar ratio of the above prepared paramagnetic polymerizable lipids with di-tricosadiynoyl phosphatidyl choline (Avanti Polar Lipids, Birmingham, Ala.) in an organic solvent methyl alcohol and chloroform (⅓) and evaporating the solvent and rehydrating with distilled water to 30 mM diacetylene (15 mM total lipid). Following sonication with a 450 W probe-tip sonicator (Virsonic 475, Virtis Corp., Gardiner, N.Y.) set at a power setting of 2½ units for 30 to 60 minutes without temperature control, the suspension of lipid aggregates was extruded ten times through two polycarbonate filters with pores of 0.1 μm diameter (Poretics, Livermore, Calif.) at 56° C. using a thermobarrel extruder (Lipex Biomembranes, Vancouver, BC). This solution was spread thinly on a petri dish in a wet ice slush and irradiated with a UV lamp, 2200 μWatt/cm held 1 cm over the solution while stirring. The solution turned orange using DAPC over the course of a one hour irradiation, due to the absorption of visible light by the conjugated ene-yne system of the polymer. The paramagnetic polymerized liposomes passed easily through a 0.2 μm sterilizing filter and were stored in solution until use. The paramagnetic polymerized lipid suspensions prepared in this manner have been found to be stable for many weeks at 4° C.

The size and shape of the paramagnetic polymerized liposomes have been ascertained by transmission electron microscopy and by atomic force microscopy. They appear as prolate ellipsoids with minor axes on the order of the membrane pore and major axes about 50 percent greater.

### Example 4

### Preparation of Chelator Lipid and Polymerized Liposomes II

The procedures of Example 3 were followed except that instead of using DAPC, pentacosadiynaic acid (PDA) was used as the filler lipid. The solution turned blue over the course of one-hour irradiation. The resulting polymerized liposomes had the same general properties as reported in Example 3.

### Example 5

### Antibody-conjugated Polymerized Liposomes I

Antibodies towards the specific immunoglobulin, anti-goat γ-IgG, were conjugated to polymerized liposomes to form antibody-conjugated polymerized liposomes for use in in vitro diagnostic applications.

Lipid components of: 60% pentacosadiynoic acid filler lipid, 29.5% chelator lipid, 10% amine terminated lipid and 0.5% biotinylated lipid were combined in the indicated amounts and the solvents evaporated. Water was added to yield a solution that was 30 mM in acyl chains. The lipid/water mixture was then sonicated for at least one hour. During sonication, the pH of the solution was maintained between 7 and 8 with NaOH and the temperature was maintained above the gel-liquid crystal phase transition point by the heat generated by sonication. The liposomes were transferred to a petri dish resting on a bed of wet ice and irradiated at 254 nm for at least one hour to polymerize. The polymerized liposomes were collected after passage through a 0.2μ filter. To form the antibody conjugated polymerized liposomes, 2.3 μg avidin was combined with 14.9 μg biotinylated antibody in phosphate buffered saline in about 1:3 molar ratio and incubated at room temperature for 15 minutes. This solution was combined with 150 μL of the above formed polymerized liposomes and incubated at 4° C. overnight to form the antibody-conjugated polymerized liposomes. The total number of antibody-conjugated polymerized liposomes in a 40 μl aliquot was found to be about 1.4×10¹¹ as determined by light scattering and theoretical calculations based on the size of the particles and protein and amount of lipid used in the preparation. The antibody-conjugated polymerized liposomes were analyzed by photon correlation spectroscopy using a Coulter N4+ submicron particle analyzer and shown to have a mean diameter of 262 nm. Then 9.6 μg of agglutinating antibody, goat IgG, was added to a 40 μl aliquot of anti-goat γ-IgG-conjugated polymerized liposomes, as prepared above, and incubated for about 1 hour. After this incubation, 53% of the antibody-conjugated polymerized liposomes had agglutinated as demonstrated by the appearance of a new group of particles with a mean diameter of 1145 nm, as determined by photon correlation spectroscopy. The antibody-conjugated polymerized liposomes thereby provide a simple and very sensitive in vitro assay for the presence of specific antigens in solution.

### Example 6

### Preparation of Chelator Lipid and Polymerized Liposomes III

Lipids containing a DTPA chelator head group were constructed as described in Storrs et al., “Paramagnetic Polymerized Liposomes: Synthesis, Characterization, and Applications for Magnetic Resonance Imaging,” J. Am. Chem. Soc. (1995) 117(28):7301-7306 incorporated herein by reference in its entirety, paragraph spanning pages 7305-7306, for compound 4 and 1b and chelated to Eu⁺³ ions and formed into polymerized liposomes at a level of 1%. A wide variety of suitable chelating agents for spectroscopically distinct ions are known to the art as, for example, as described in U.S. Pat. Nos. 4,259,313; 4,859,777; 4,801,504; 4,784,912; and 4,801,722. The Europium-labelled polymerized liposomes were serially diluted with buffer and detected using time-resolved fluorescence spectroscopy, detecting Eu⁺³ labeled polymerized liposomes down to concentrations of 10-21 molar in an ELISA-based system.

### Example 7

### Preparation of Polymerized Liposomes IV

Polymerized liposomes based upon pentacosadiynoic acid were constructed having a negative charge. No exogenous fluorescent probes were used and only the intrinsic fluorescence of the polymerized liposomes, emission at 530-680 nm, was relied upon for detection. The polymerized liposomes were incubated with endothelial cells expressing P-Selectin, a protein that binds charged entities, and then analyzed using flow cytometry. Flow cytometry detected the polymerized liposomes adhered to the endothelial cells.

### Example 8

### Preparation of Polymerized Liposomes V

A lipid containing a fluorophore head group, such as, for example, Texas Red, was constructed. Suitable lipids are, for example, PDA (PEG)₃-NH₂/carboxylic acids and hydrazine derivatives and suitable fluorophore head groups are, for example, Texas Red and FITC. This material was incorporated into polymerized liposomes at a level of 0.5%. 200 μg Texas Red sulfonyl chloride in acetonitrile was added to 600 μl polymerized liposomes, 30 mM in acyl chain, on 0.01M sodium bicarbonate buffer, pH 9, and reacted at room temperature for 2 hours. The labeled polymerized liposomes were then purified by gel filtration (Sephadex G-25, Sigma, St. Louis, Mo.) using PBS as eluent. An anti-ICAM-1 antibody was then attached to the Texas Red labelled polymerized liposomes in the same manner as described in Example 4 and then incubated with activated endothelial cells expressing ICAM-1 and analyzed using fluorescent microscopy. Using this approach, 10⁵ to 10⁶ Texas Red molecules can be linked to each antibody resulting in dramatic increase in sensitivity of the assay. The antibody conjugated polymerized liposomes can be easily seen bound to the activated endothelium, thus simplifying the methodology for assaying cell surface glycoproteins.

### Example 9

### Antibody-conjugated Polymerized Liposomes II

To conjugate monoclonal antibodies to paramagnetic polymerized liposomes, paramagnetic polymerized liposomes containing biotinylated lipids were constructed. Avidin, a biotin binding protein, was then used to bridge biotinylated antibodies to biotin on the particle surface. Alternatively, anionic polymerized liposome particles may be constructed and antibodies conjugated to cationic proteins, such as avidin, are then exchanged onto the particles.

Lipid components of: 60% pentacosadiynoic acid filler lipid, 29.5% Gd⁺³ chelator lipid, 10% amine terminated lipid and 0.5% biotinylated lipid were combined in the indicated amounts and the solvents evaporated. Water was added to yield a solution 30 mM in acyl chains. The lipid/water mixture was then sonicated for at least one hour. During sonication, the pH of the solution was maintained between 7 and 8 with NaOH and the temperature was maintained above the gel-liquid crystal phase transition point by the heat generated by sonication. The liposomes were transferred to a petri dish resting on a bed of wet ice and UV irradiated at 254 nm for at least one hour to polymerize. The paramagnetic polymerized liposomes were collected after passage through a 0.2 μm filter. The resulting paramagnetic polymerized liposomes were dark blue and exhibited absorption bands at 544 nm, 588 nm and 638 nm (λₘₐₓ). Gentle heating turned the paramagnetic polymerized liposomes red having absorption maxima at 498 nm and 538 nm. All paramagnetic polymerized liposomes used in this study were converted to the red form.

To form antibody conjugated paramagnetic polymerized liposomes, 2.3 μg avidin was combined with 14.9 μg biotinylated antibody in phosphate buffered saline in about 1:3 molar ratio and incubated at room temperature for 15 minutes. This solution was combined with 150 μL of the above formed paramagnetic polymerized liposomes, 5.6 mM in acyl chains, and incubated at 4° C. overnight to form the anti-cell adhesion molecule antibody-avidin conjugation to the biotinylated polymerized liposomes.

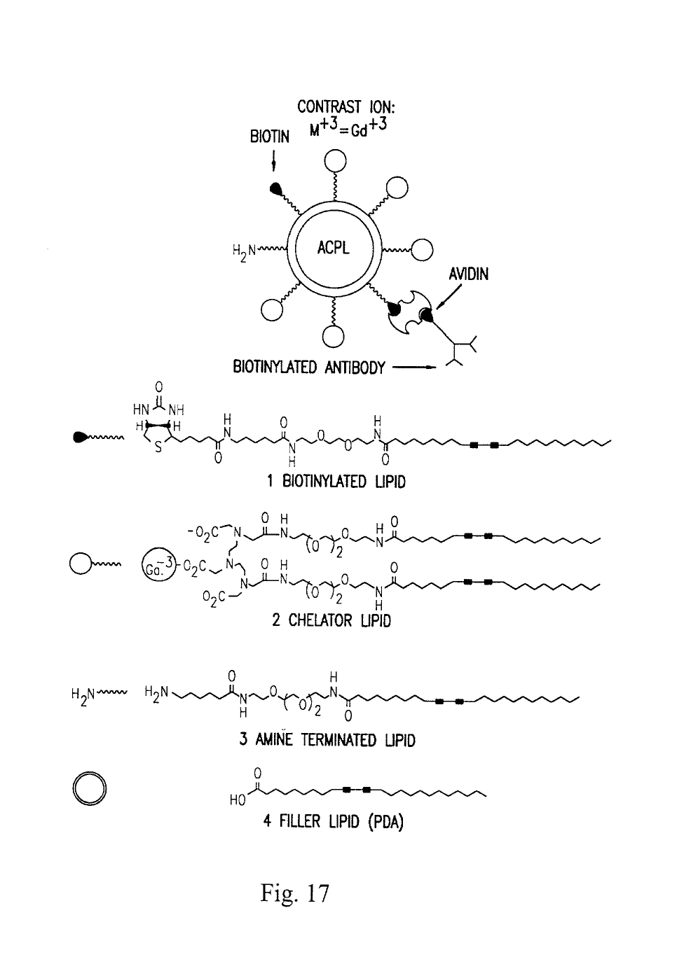

FIG. 17 schematically shows the antibody-conjugated paramagnetic polymerized liposome (ACPL) formed as described above.

### Example 10

### Antibody-conjugated Polymerized Liposomes III

Attachment of the monoclonal antibodies to the biotinylated paramagnetic polymerized liposomes, as prepared in Example 9, was confirmed using gel electrophoresis and immunodetection techniques.

For gel electrophoresis, samples were run on 0.65% agarose gels under non-denaturing conditions, running buffer 25 mM Tris, 190 mM glycine, pH 7.5. Gels were fixed in a solution of 45% methanol and 10% acetic acid for 15 minutes, rinsed overnight in water, incubated in 1% rabbit normal serum for 2 hours at room temperature, and incubated overnight at 4° C. with a 1:1000 dilution in PBS of alkaline phosphatase-conjugated antibodies against avidin (Sigma) or γ-immunoglobulin (Victor Laboratories, Burlingame, Calif.). After rinsing in several changes of PBS, gels were incubated at room temperature in the enzyme substrate, 5-bromo 4-chloro 3-indolyl phosphate 0.16 mg/ml and nitro blue tetrazolium 0.32 mg/ml (Sigma) in 0.1 M NaCl, 0.1 M Tris, 50 mM MgCl₂, pH 9.5, until the gel was adequately developed. The reaction was stopped by rinsing in 1 mM EDTA. The paramagnetic polymerized liposomes contain a chromophore and were therefore visible without staining.

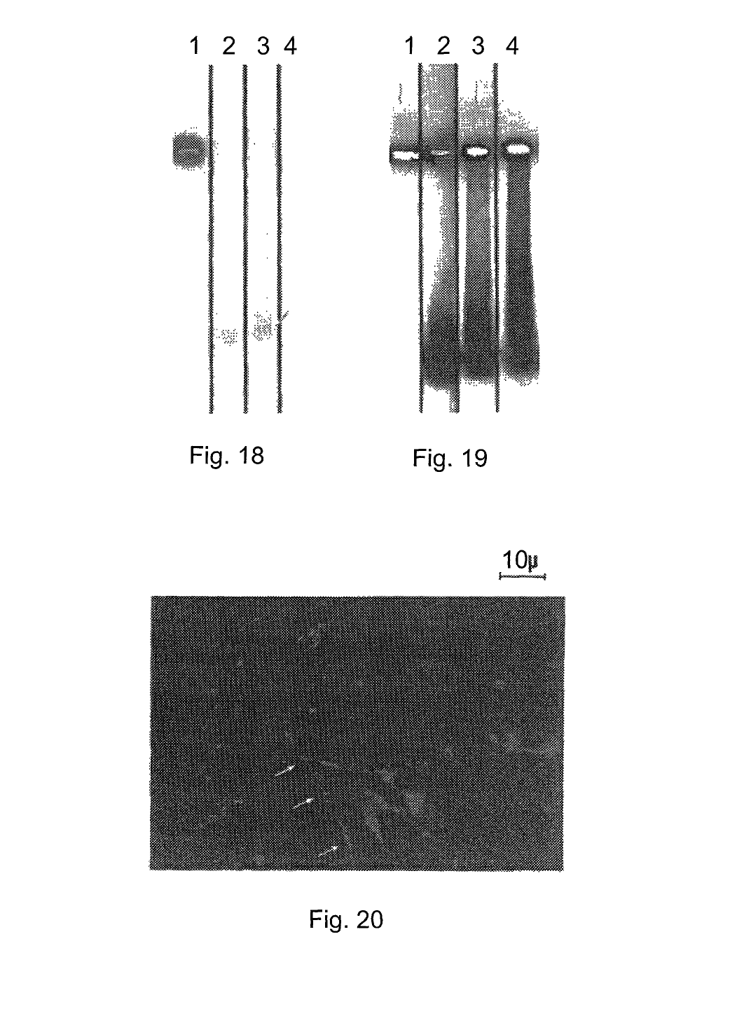

Gel electrophoresis, using anti-avidin alkaline phosphatase, in FIG. 18, showed in Lane 1 intense staining of 0.5 μg avidin, which, apparently at its isoelectric point, moved slowly from the loading well. Lane 2 showed a 5 μL sample of paramagnetic polymerized liposomes moved as a discrete band toward the positive pole. A solution of approximately 1:3 molar ratio of avidin, 4 μg, and unbiotinylatled anti-CAM antibody, 26.25 μg, was incubated in a total volume of 60.5 μL, PBS at 4° C. for 48 hours. A 3.2 μl aliquot of this solution was added to 16 μL of paramagnetic polymerized liposomes and incubated for approximately 1 week at 4° C. A 5 μL sample of paramagnetic polymerized liposomes pre-incubated with avidin and unbiotinylated anti-CAM antibody, as prepared above, showed, in Lane 3, avidin co-migrated with the liposome band, indicating the avidin was bound to the surface of the paramagnetic polymerized liposomes. No free avidin was detected near the well. Antibody-conjugated paramagnetic polymerized liposomes were prepared in the manner described above, except that biotinylated anti-CAM antibody was used, allowing conjugation of the antibody to the avidin-paramagnetic polymerized liposome complex to form antibody-conjugated paramagnetic polymerized liposomes. A 5 μL sample of the biotinylated anti-CAM antibody-conjugated polymerized liposomes showed, in Lane 4, no free avidin detected indicating that the avidin was bound to the paramagnetic polymerized liposomes. However, no avidin band appeared with the liposomes, suggesting that antibody conjugation to the particle surface sterically hindered binding of the anti-avidin alkaline phosphatase immunodetection antibody to the complex.

For immunodetection by anti-IgG alkaline phosphatase to assess antibody binding to the paramagnetic polymerized liposomes, paramagnetic polymerized liposome preparations and antibody/avidin incubations were performed as described above for the anti-avidin alkaline phosphatase immunodetection. FIG. 19 shows a 2.5 μg aliquot of biotinylated anti-CAM antibody moved as a distinct band in Lane 1 toward the negative pole. A 5 μL sample of paramagnetic polymerized liposome, as above, showed in Lane 2, movement toward the positive pole, being visible due to its intrinsic chromophore. A 5 μl sample of paramagnetic polymerized liposomes pre-incubated with avidin and unbiotinylated antibody, 2.2 μg total antibody, exhibited a free antibody band, in Lane 3, indicating that unbiotinylated antibody did not bind with the avidin-paramagnetic polymerized liposome complex. A 5 μl sample of paramagnetic polymerized liposomes pre-incubated with avidin and biotinylated antibody, 2.2 μg total antibody, in Lane 4, exhibited no detection of a free antibody band, demonstrating conjugation of the biotinylated antibody to the avidin-paramagnetic polymerized liposomes forming antibody-conjugated paramagnetic polymerized liposomes.

This Example shows that the antibody-conjugated paramagnetic polymerized liposome is functional in a competitive ELISA assay. Anti-ICAM-1 antibody-conjugated paramagnetic polymerized liposomes incubated on ELISA plates coated with soluble ICAM-1 demonstrated inhibition of free monoclonal anti-ICAM-1 antibody binding.

### Example 11

### Cell-binding Assays Using Fluorescently-tagged Antibody-conjugated Paramagnetic Polymerized Liposomes

Cell-binding assays using fluorescently-tagged antibody-conjugated paramagnetic polymerized liposomes were conducted to show that the anti-ICAM-1 antibody-conjugated paramagnetic polymerized liposomes could recognize antigens in vitro. Paramagnetic polymerized liposomes, as prepared in Example 9, were coupled to Texas Red fluorophore (Pierce, Rockford, Ill.). 200 μg Texas Red sulfonyl chloride in acetonitrile was added to 600 μl paramagnetic polymerized liposomes, 30 mM in acyl chain, in 0.1 M sodium bicarbonate buffer, pH 9, and reacted at room temperature for 2 hours. The labeled paramagnetic polymerized liposomes were then purified by gel filtration (Sephadex G-251 Sigma, St. Louis, Mo.) using PBS as eluent. Fluorescent paramagnetic polymerized liposomes were then conjugated to anti-ICAM-1 antibodies as described in the prior example.

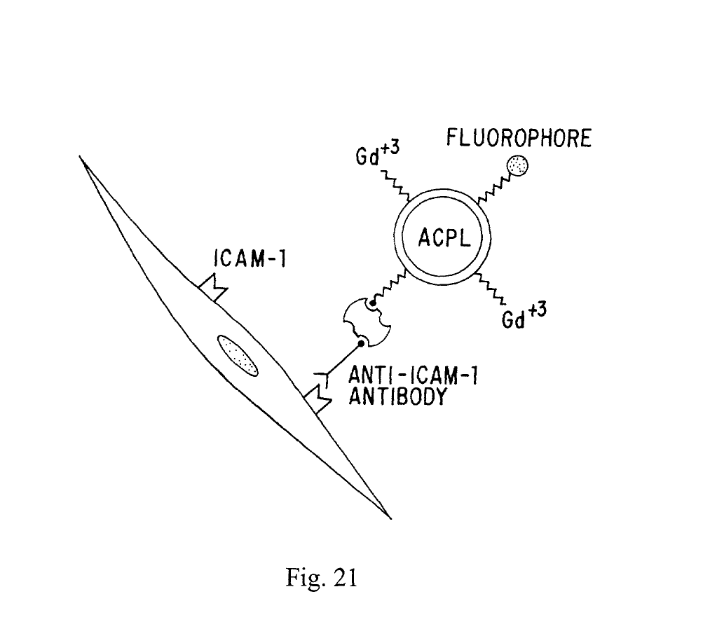

Endothelial cells, bEnd 3, were plated onto 100 mm plastic petri dishes and grown until confluent. Cells were stimulated with 1 μg/ml bacterial lipopolysaccharide about 24-48 hours prior to use to elicit expression of ICAM-1. Unstimulated cells constitutively expressing only low levels of adhesion molecules were used as controls. Media was aspirated from cells and the plates were rinsed with Hank's balanced salt solution for 30 minutes, washed three times with PBS and then divided in 1 cm² wells. The wells were pre-incubated with 0.5% bovine serum albumin in PBS for approximately 3 hours at room temperature following which aliquots of 50 μl each of 1:100 and 1:1000 dilutions of antibody-conjugated paramagnetic polymerized liposomes were added to cover the wells. Antibody-conjugated paramagnetic polymerized liposomes were incubated with the cells for 2 hours at room temperature and then washed two times for five minutes with 0.5% BSA-PBS and four times for five minutes with PBS. Using fluorescence microscopy, fluorescently tagged anti-ICAM-1 antibody-conjugated paramagnetic polymerized liposomes were seen bound to the cultured endothelial cells stimulated with bacterial lipopolysaccharide to elicit ICAM-1 expression, outlining the morphology of individual cell membranes, as shown in FIG. 20. This binding is shown schematically in FIG. 21. No binding of fluorescent antibody-conjugated paramagnetic polymerized liposomes to stimulated cells was observed when a non-specific anti-immunoglobulin antibody was substituted for anti-ICAM-1. Similarly, unstimulated cells that express only low levels of ICAM-1 did not bind anti-ICAM-1 fluorescent antibody-conjugated paramagnetic polymerized liposomes.

### Example 12

### In vivo Targeting of Endothelial CAMs with Antibody-conjugated Paramagnetic Polymerized Liposomes

To show that antibody-conjugated paramagnetic polymerized liposomes could both successfully target endothelial CAMs in vivo and also provide substantial magnetic resonance image contrast enhancement, a well-documented model of cerebral inflammation in mice was examined.

Experimental autoimmune encephalitis is an ascending encephalomyelitis characterized by an intense perivascular lympho-/monocytic inflammatory process in the central nervous system white matter, primarily the cerebellum, brain stem and spinal cord. This system is of clinical interest as an animal model for multiple sclerosis and the nature of the receptors involved in inflammatory cell trafficking in experimental autoimmune encephalitis have been well investigated. ICAM-1 expression on the experimental autoimmune encephalitis mouse brain microvasculature has been shown to be upregulated at the onset of clinical disease. The ICAM-1 receptor mediates the attachment of leukocytes to inflamed endothelium and is present on both activated leukocytes and stimulated endothelium of capillaries and venules throughout the central nervous system. Its expression is not limited to vessels involved by inflammatory infiltrates. Histologic studies have previously shown that the blood-brain barrier maintains integrity during the onset of disease and for 48 hours after paralysis is apparent. Prior magnetic resonance and fluorescence microscopy studies of liposome transit across the blood-brain barrier in acute experimental autoimmune encephalitis guinea pigs have shown that liposomes were unable to penetrate compromised blood-brain barrier and enter brain parenchyma. Therefore, the ICAM-1 receptor was targeted in the early phase of its upregulation in experimental autoimmune encephalitis, when expression of ICAM-1 is increased ten-fold.

Fluorescently labeled anti-ICAM-1 antibody-conjugated paramagnetic polymerized liposomes were shown in vivo to bind to cerebellar vasculature of mice with grade 2 experimental autoimmune encephalitis by showing location of the particle as seen by high resolution magnetic resonance could be confirmed with fluorescence microscopy.

Experimental autoimmune encephalitis was induced in SJL/J mice according to a proteolipid protein immunization protocol. When clinical signs of grade 2 disease were apparent, tail paralysis and limb weakness, the fluorescent anti-ICAM-1 antibody-conjugated paramagnetic polymerized liposomes, as prepared in the prior example, were injected via a tail vein, 10 μl/g representing 1.2 mg/kg Gd⁺³ and 890 μg antibody/kg, and allowed to recirculate for 24 hours. Mice were then sacrificed and perfused with PBS. The brains were removed and cut in half sagittally, one half frozen for direct fluorescence microscope analysis of 10 μm thin sections and the other half fixed in 4% paraformaldehyde in PBS, pH 7.4, and used for high resolution magnetic resonance imaging.

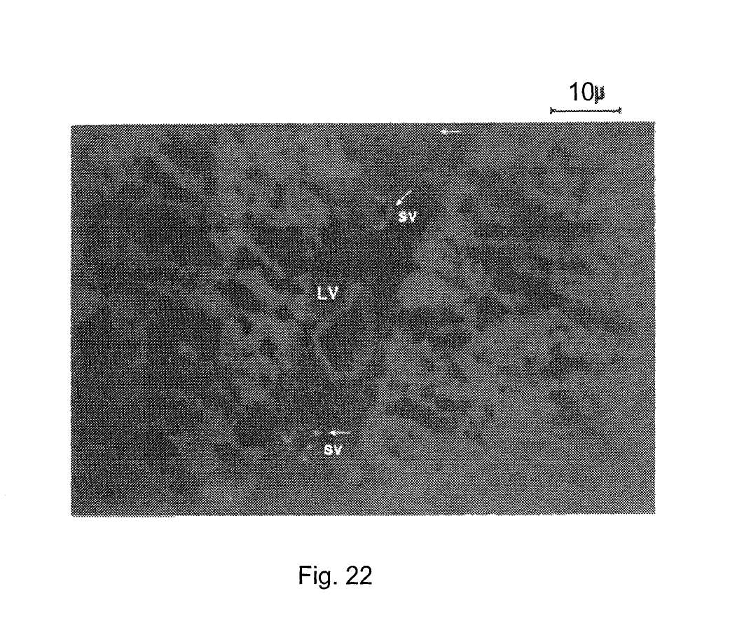

In three separate tests, a total of seven diseased mice were injected with fluorescent anti-ICAM-1 antibody-conjugated paramagnetic polymerized liposomes and all were shown to be positive for the antibody conjugated-polymerized liposome binding to central nervous system vasculature by fluorescence microscopic analysis of cerebellum, brainstem and spinal cord. FIG. 22 is a typical fluorescence micrograph of mouse cerebellum counterstained with haematoxylin showing multiple vessels surrounded by an inflammatory infiltrate. Anti-ICAM-1 antibody-conjugated paramagnetic polymerized liposomes, indicated by arrows, are seen by fluorescence to be bound to small capillaries (SV), but not bound to large central arteriole (LV) which is seen to be negative for fluorescence. This is consistent with expression of ICAM-1 which is upregulated on endothelium of venules and capillaries, but not expressed on arterioles or larger vessels. It was also noted that fluorescent anti-ICAM-1 polymerized liposomes bound to microvessels that are not associated with inflammatory infiltrates, which is consistent with histological findings of ICAM-1 expression on both infiltrated and non-infiltrated vessels.

Six controls: three healthy animals injected with anti-ICAM-1 antibody-conjugated paramagnetic polymerized liposomes; two diseased animals administered anti-trinitrophenol antibody-conjugated paramagnetic polymerized liposomes, and one diseased animal administered anti-Vβ11 T-cell receptor antibody-conjugated paramagnetic polymerized liposomes, targeted to an antigen not expressed in the SJL/J mouse, were all found by fluorescence microscopy to show no polymerized liposome binding.

### Example 13

### Magnetic Resonance Imaging of Anti-ICAM-1 Antibody-conjugated Paramagnetic Polymerized Liposomes

High-resolution magnetic resonance images were made of the complementary half of two mouse brains from mice having grade 2 experimental autoimmune encephalitis used in the previous example containing anti-ICAM-1 antibody-conjugated paramagnetic polymerized liposomes. High resolution T1 and T2-weighted images of the intact half brains were obtained by using a 9.4T MR scanner (General Electric) using 3DFT spin echo pulse sequences.

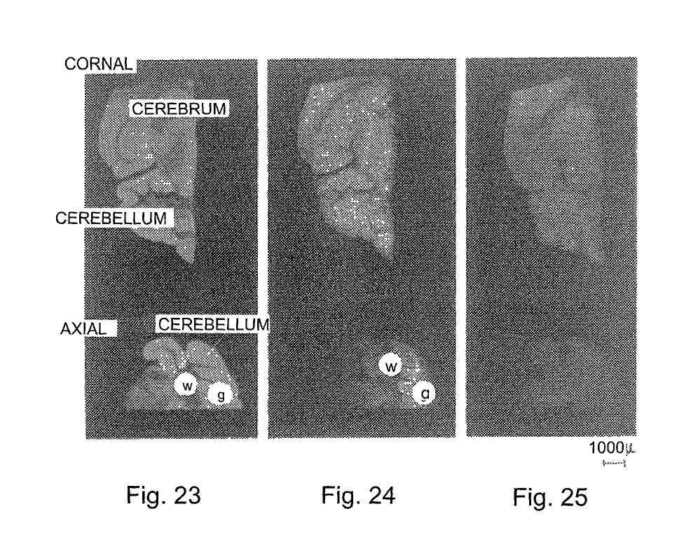

Parameters for T1-weighted images were TR 200 ms, TE 4 ms, 1 NEX, matrix 256×256×256, and a field of view of 1 cm, resulting in a voxel size of approximately 40 μm in each dimension. T1-weighted acquisitions times were approximately 7 hours per scan. T2-weighted parameters were TR 1000 ms, TE 20 ms, 8 NEX, matrix 256×256×256. T2-weighted scan times were approximately 12 hours. FIG. 23 shows a T2-weighted scan of an experimental autoimmune encephalitis mouse, without injection of polymerized liposomes, cerebrum (coronal) and cerebellum (axial) to define normal anatomy. FIG. 24 shows a representative slice from a T1-weighted scan of an autoimmune encephalitis mouse injected with anti-ICAM-1 antibody-conjugated paramagnetic polymerized liposomes. Diffuse perivascular enhancement is seen throughout the brain, in the cerebellum and cerebrum, lending particularly significant contrast between the meagerly vascularized cerebellar white (W) and the highly vascular grey (g) matter. FIG. 25 shows a representative slice from a T1-weighted scan of a healthy mouse similarly injected with anti-ICAM-1 antibody-conjugated paramagnetic polymerized liposomes showed no enhancement.

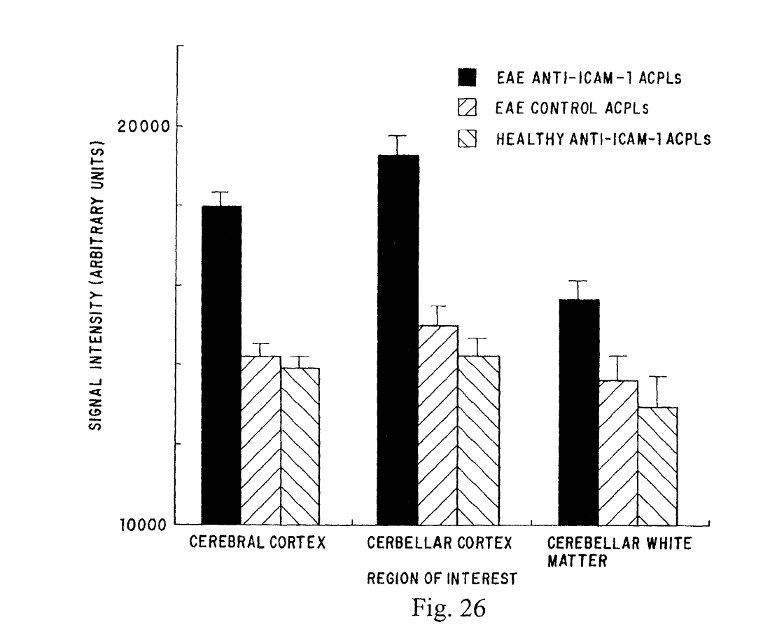

Signal intensity measurements were made using the image analysis program Voxel View/Ultra 2.2 (Vital Images, Inc., Fairfield, Iowa). For each mouse brain, three slices were chosen for analysis. For each slice, the signal intensity of cerebral gray, cerebellar gray, and cerebellar white matter was determined by manually drawing at least five large region-of-interest paths within each of these tissues. Signal intensity measurements from the three slices were averaged to give a mean signal intensity value for each tissue type, means weighted according to standard deviation of individual signal intensity values. The differences in tissue signal intensities between mouse brains were assessed using the two-tailed Student's t-test. The statistical significance level was set at P<0.05. The results are shown in FIG. 26. Compared to the controls, the magnetic resonance scans of the experimental autoimmune encephalitis infected mice injected with anti-ICAM-1 antibody-conjugated paramagnetic polymerized liposomes showed substantial increases in magnetic resonance signal intensity of about 32% in the cerebellar, 28% in the cerebral cortex and, to a lesser extent, about 18% in the cerebellar white matter. As a result of the enhanced gray matter signal, contrast between gray and white matter was improved. This was particularly pronounced in the cerebellum which was actively affected by experimental autoimmune encephalitis.

The above examples have demonstrated that antibody-conjugated paramagnetic polymerized liposomes can be delivered to cell adhesion molecules upregulated in disease. This provides a new target-specific magnetic resonance contrast enhancement agent for providing in vivo imaging studies of specific targeted physiological activities, such as, for example, endothelial antigens involved in numerous pathologies.

### Example 14

### Preparation of Chelating Polymerized Vesicles (CPVs)

To a 100 mL round bottom flask was added 11 mL (220 mg, 240 μmol) of BisT-PC lipid 6 (FIG. 29)at 20 mg/mL chloroform and 3 mL (15 mg, 11 μmol) DTTA lipid 5 (FIG. 29) at 5 mg/mL chloroform. The chloroform was removed at 60° C. by rotary evaporation. Water (10 mL) was added and the solution was frozen on a dry ice/acetone mixture until solid. The pH was adjusted to 8 by adding 20 μL aliquots of 0.5 M NaOH. The freeze thaw process was repeated three times or until a translucent solution was obtained. This solution was passed through a 30 nm polycarbonate filter in a thermal barrel extruder (Lipex Biomembranes, Inc.) heated at 80° C. and pressurized with argon to 750 PSI. Vesicle size was determined by dynamic light scattering (Brookhaven Instruments). Polymerization of diacetylene containing lipids was achieved by cooling the vesicles to 2-4° C. in a 10×1 polystyrene dish (VWR) and irradiating with UV light using a hand-held UV illuminator at approximately 3.8 mW/cm². The optical density at 500 nm for the orange vesicles was approximately 0.4 AU at 1 mg/mL of vesicle in water. Yellow vesicles were prepared by polymerization at 12° C. and the optical density was 1 AU at 1 mg/mL vesicle in water. Liposomes containing chelating lipid 5, cholesterol, and egg phosphatidylcholine (5/28/67 mole percent) were prepared without polymerization.

### Example 15

### Metal Binding to Chelating Vesicles

Yttrium-90 chloride or indium-111 chloride (10-20 mCi) in 50 mM HCl was diluted with 50 mM citric acid (pH 4) to give a solution that was 50 mCi/mL. To 90 μL of vesicle solution in 50 mM histidine buffer containing 5 mM citrate at pH 7 was added 10 μL of isotope solution containing 100-200 μCi. The solution was incubated at room temperature for 30 minutes and added to a 100K MWCO spin filter cartridge (Nanosep), which was placed in a table top centrifuge. After spinning at 3000 rpm for 90-120 minutes, the isotope was quantified using a Capintec CRC-15R dose calibrator. The filter portion of the cartridge that contains the vesicle-isotope complex was removed, and the remaining unbound isotope was quantified. These values were used to calculate the percent metal bound, or the amount of isotope bound per mg of vesicle.

### Example 16

### ICP-MS

Yttrium-90 was determined by measuring the decay product, zirconium-90, by inductively coupled plasma mass spectrometry (ICPMS) with a Perkin Elmer ELAN 6100 DRC. Yttrium samples or samples in an identical matrix without yttrium were diluted as described above and were further diluted in triply distilled water containing 5% concentrated nitric acid.

### Example 17

### Determination of Chelator Concentration

The chelator concentration was determined using constant yttrium-90 (100 μCi) in the presence of variable yttrium-89 to give total yttrium concentrations of 20-1000 μM where yttrium-90 is ≈1 μM. Briefly, yttrium-90 (20 mCi in 100 μL of 50 mM HCl) or yttrium-89 chloride in 50 mM HCl was diluted with 50 μL of 50 mM HCl and 350 μL of 50 mM sodium citrate. In a typical assay, yttrium-89 solution (100-200 μCi, 4 μL), yttrium-89 solution (5 μL), 100 mM histidine buffer containing 10 mM sodium citrate pH 7.4 (25 μL), water (16 μL), and 2 mg/mL CPV in 50 mM histidine buffer containing 5 mM sodium citrate at pH 7.4 (50 μL). The yttrium bound to the vesicles was determined as described above, and the chelator concentration was determined by extrapolation from a plot of % yttrium bound vs. yttrium concentration. Alternatively, the chelator concentration was determined by adding variable amounts of yttrium-89 to vesicles followed by yttrium-90.

### Example 18

### Attachment of Antibodies to Vesicles

Antibodies were attached to chelating vesicles prepared as in Example 15 as described in this example. To an aqueous solution of vesicles (25 mg/mL, 40 μL) was added 500 mM borate buffer at pH 8 (10 μL), Vitaxin (5 mg/mL, 5 μL), water (42.5 μL), and EDAC (200 mM, 2.5 μL). The solution was incubated at room temperature for 18 h and purified from unreacted antibody by size exclusion chromatography on a column of Sepharose CL 4B equilibrated with 10 mM HEPES buffer at pH 7.4. Fractions were collected and assayed for antibody by ELISA as described below. Fractions containing vesicles were identified by UV/VIS spectroscopy.

### Example 19

### Attachment of Peptides to Vesicles

Peptides were attached to vesicles as described in this example for peptide Gly-Arg-Gly-Asp-Ser (GRGDS). To an aqueous solution of vesicles (20 mg/mL, 100 μL) was added water (70 μL), 500 mM MOPS buffer at pH 7 (10 μL), and peptide GRGDS at 25 mM (10 μL). EDAC (8 μL, 500 mM) was added and the solution was incubated for 18 h. The conjugates were purified by dialysis (10K MWCO) or by size exclusion chromatography as described above. RGD peptide couplings were monitored by HPLC at 214 nM with a TosoHaas TSK G2500 PW×1 column using 50 mM borate buffer containing 200 mM sodium chloride at pH 8.

### Example 20

### ELISA for Antibody-vesicle Conjugates

The presence of antibodies on the vesicles was verified by ELISA as described in this example. For rat or mouse antibodies, the corresponding anti-species antibody was used. 96-well plates were coated with goat anti-human Fc (γ) antibodies (KPL) at 2 μg/mL in PBS buffer overnight. The wells were washed 3 times with 300 μL of wash solution (Wallac Delfia Wash) and blocked with 200 μL of milk blocking solution (KPL) for 1 h at RT. Antibody-vesicle conjugates (50 μL) were added at a concentration of 1-100 μg/mL in 50 mM HEPES buffer at pH 7.4. Following a 1 h incubation at RT, the wells were washed 3 times. Goat anti-human Fc (γ) antibody-HRP conjugate (KPL) in milk blocking solution at 1 μg/mL was added. Following a 1 h incubation at RT, the wells were washed twice and Lumiglo chemiluminescent substrate (KPL, 50 μL) was added. After a 1 minute incubation, the signals were monitored using a Wallac Victor luminescence reader.

### Example 21

### In-vitro Targeting of Antibody- and Peptide-CPV-⁹⁰Y Conjugates

Targeting was demonstrated in-vitro using a radiometric binding assay specific to the αᵥβ₃ integrin that requires an intact tripartite complex consisting of antibody or peptide, CPV, and yttrium-90. Briefly, 96 well plates coated with the αᵥβ₃ integrin (Chemicon International, Inc.) were blocked with BSA. Samples of rabbit serum or buffer containing 0-100 micrograms/mL of the anti-αᵥβ₃ integrin antibody-liposome-yttrium-90 complex, or corresponding peptide complex, were added and incubated for 1 hour at room temperature. The plate was washed three times with PBST buffer and the yttrium-90 was measured using a Microbeta scintillation counter (Wallac).

### Example 22

### Cell Adhesion Inhibition Assay

The inhibition of cell adhesion was performed using a modified protocol (A. Howlett, Ed., Integrin Protocols, vol. 129 (Humana Press, Totowa, 1999)). 96-well plates were coated with 100 μL of fibrinogen at 1 μg/mL in PBS at 4° C. overnight. The solution was removed and 1% BSA in PBS was added followed by a 1 hour incubation at 37° C. This solution was removed and the plates were washed with 200 μL PBS (3×). M21 human melanoma cells grown to confluency in RPMI 1640 growth media containing 10% FBS, glutamine, penicillin, and streptomycin were washed 2× with PBS and detached by incubating in PBS containing 2 mM EDTA and 1% glucose. The cells were pelleted by centrifugation, washed 2× in assay medium (RPMI 1640 containing 20 mM HEPES pH 7.5, 1 mM MgCl₂, 1 mM CaCl₂, 0.25 mM MnCl₂, and 0.1 BSA), and suspended at 660,000 cells/mL. Vitaxin-CPVs or GRGDS-CPVs in assay medium were diluted and 50 μL was added to each well followed by 50 μL of cells solution. After incubation at 37° C. in 5% CO₂ for 1 h, the plates were washed 3× with 200 μL of PBS and 100 μL of 70% ethanol was added. After 1 h, the ethanol was removed and 0.2% crystal violet was added for 30 min. The plates were washed 4× with 200 μL of deionized water and 100 μL of 1% SDS was added for 60 minutes. The absorbance at 590 nm was measured using a Wallac Victor plate reader. IC50s were determined using the Kaleidagraph application.

### Example 23

### In vivo MR Studies of Antibody-conjugated Imaging of Anti-integrin Antibody-conjugated Paramagnetic Polymerized Liposomes

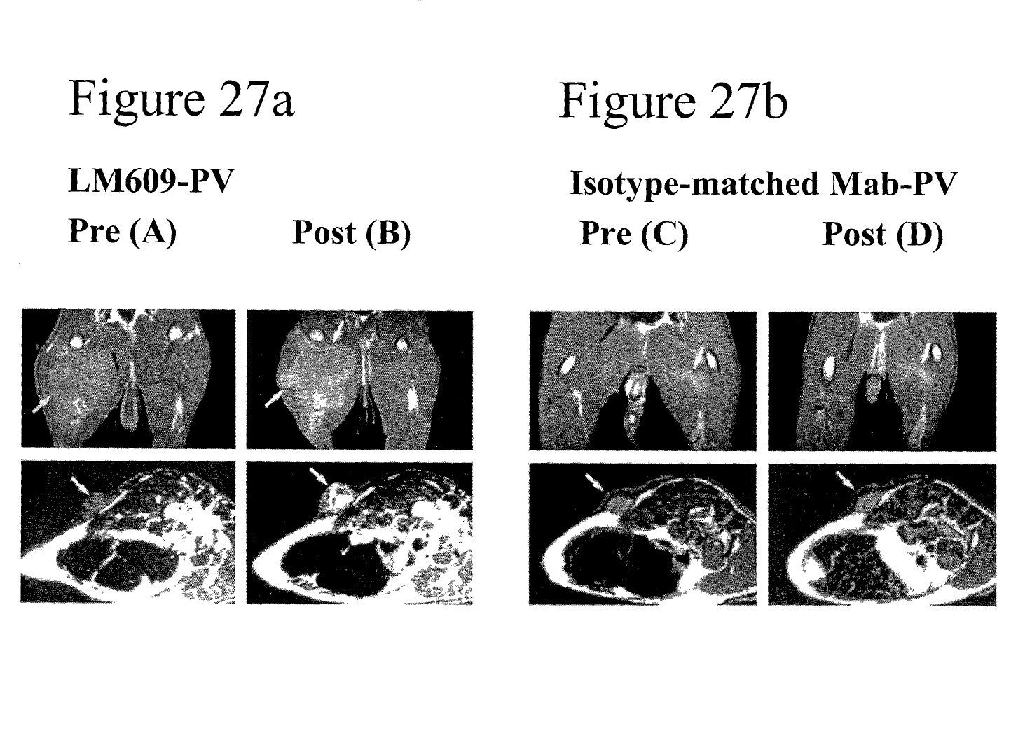

Murine antibodies against the αᵥβ₃ integrin (LM609) were conjugated to polymerized diacetylene vesicles (PVs) to form Ab-PVs and evaluated in a rabbit tumor model (Vx2 carcinoma) that has previously shown upregulation of the integrin on the vasculature. Vx2 carcinoma cells were inoculated into the thigh muscle or placed subcutaneously in New Zealand white rabbits. The rabbits were closely monitored until a palpable tumor was established. For in vivo MR studies, rabbits with palpable tumors (approximately 1-3 cm in diameter) were injected intravenously with either 5 ml/kg (approx. 30 mM in total lipid) anti-αᵥβ₃ (LM609)-labeled AbPVs (1 mg antibody/kg, 0.005 mmol Gd⁺³/kg) or control AbPVs with isotype matched control antibodies. MR imaging was performed using a 1.5 T GE Signa MR imager using an extremity coil and the following imaging parameters: TR=300 ms, TE=18 ms, NEX=2, FOV=16 cm, 256×256 matrix, slice thickness=3 mm. MR images were obtained immediately prior to contrast injection and at immediate, 30 minutes, 1 hour and 24 hours post-contrast injection in the coronal plane. The rabbits were euthanized immediately following the last MR imaging experiment and the tumor tissues were harvested for immunohistochemical studies. FIG. 27 illustrates the MR findings of a Vx2 carcinoma carrying rabbit injected with LM609-labelled AbPVs. At immediate, 30 minutes and 1-hour post-contrast injection no noticeable enhancement of the tumor or tumor margin occurs as compared to the pre-contrast image (FIG. 27A, Pre(A)), whereas at 24 hours post-contrast injection (FIG. 27B, Post(B)), enhancement of the tumor margin is clearly visible.

Isotype-matched controls showed low contrast enhancement in 24-hour post-contrast injection in both tumor models (compare images Pre(C) to Pre(D) in FIG. 27B).

### Example 24

### Nuclear Scintigraphy of the Vx2 Carcinoma in Rabbits

Radiolabeling of CPVs and CPV conjugates was achieved by labeling with ¹¹¹InCl₃(DuPont NEN) as described above to obtain doses between 0.25 and 0.5 mCi/kg and 10 mg of CPV/kg. Rabbits bearing the Vx2 carcinoma in the thigh muscle (D. A. Sipkins, et al., Nat Med 4, 623-6 (1998)) were weighed and anesthetized with a mixture of Ketamine (35 mg/kg) and Xylazine (4 mg/kg). Radiolabeled CPV solution (approximately 2 mL) was administered via the marginal ear vein. Scans were obtained immediately after i.v. administration and at 8, 24, 48, and 72 hours post-injection. Planar images of the upper torso and the hindquarters were collected for 15 minutes on a gamma camera equipped with a medium-energy collimator and a 20% energy window set at 174-247 keV.

### Example 25

### Receptor Targeted Molecular Radioimmunotherapy

The use of Ab-PVs as a platform to develop receptor-targeted molecular radioimmunotherapy for tumor angiogenesis was also studied. By designing a particle carrying a high payload of yttrium-90 (⁹⁰Y) and LM609, the mouse MAb that binds the integrin αᵥβ₃ that is upregulated in tumor-induced angiogenesis, a radioimmunotherapy approach to ablating tumor neovasculature was investigated. Vx2 carcinoma cells were implanted in the thighs of 36 New Zealand white rabbits. The tumor growth was monitored by serial MR imaging of the rabbits. After 7 days of tumor growth, a single bolus injection of therapy (4 mg polymerized vesicle/kg, 0.1 mg/kg MAb and 0.6 mCi/kg of ⁹⁰yttrium) was injected intravenously. Targeted polymerized nanoparticles with ⁹⁰Y reduced tumor growth rates by approximately 50% compared to untreated controls. MAb alone and polymerized vesicle alone had no effect on tumor growth. The ⁹⁰Y was required since no tumor growth effects were observed with the MAb conjugated vesicle without radioactivity. Other controls included the untargeted vesicle with and without ⁹⁰Y and MAb-targeted vesicle without ⁹⁰Y, all of which showed little or no effect on tumor growth. These results suggest that radioimmunotherapy using a high yttrium-payload on polymerized vesicles labeled with a MAb-targeting tumor angiogenesis is a viable strategy for the treatment of solid tumors.

All references, publications, patents and patent applications mentioned herein are hereby incorporated by reference herein in their entirety.

Although the foregoing invention has been described in some detail by way of illustration and example for purposes of clarity and understanding, it will be apparent to those skilled in the art that certain changes and modifications may be practical. Therefore, the description and examples should not be construed as limiting the scope of the invention, which is delineated by the appended claims.

What is claimed is:

1. A targeted therapeutic agent comprising:
a targeting entity which binds to a site of pathology;
a linking carrier; and
a therapeutic entity.

2. The targeted therapeutic agent of claim 1, wherein the targeting entity binds to neovasculature associated with a site of pathology.

3. The targeted therapeutic agent of claim 1, wherein the targeting entity binds to an endothelial receptor or a tissue accessible through a bodily fluid.

4. The targeted therapeutic agent of claim 1, wherein the targeting entity binds to a receptor upregulated in a tissue or cell adjacent to or in a bodily fluid.

5. The targeted therapeutic agent of claim 1, wherein the site of pathology is a tumor.

6. The targeted therapeutic agent of claim 1, wherein the targeting entity is an antibody.

7. The targeted therapeutic agent of claim 6, wherein the antibody is directed against the marker αᵥβ₃.

8. The targeted therapeutic agent of claim 6, wherein the antibody is selected from the group consisting of an anti-ICAM-1 antibody, an LM609 antibody and a Vitaxin antibody.

9. The targeted therapeutic agent of claim 1, wherein the targeting entity is a peptide.

10. The targeted therapeutic agent of claim 9, wherein the peptide contains an RGD amino acid sequence.

11. The targeted therapeutic agent of claim 1, wherein the targeting entity is a small molecule ligand.

12. The targeted therapeutic agent of claim 1, wherein the targeting entity is a carbohydrate.

13. The targeted therapeutic agent of claim 1, wherein the linking carrier is selected from the group consisting of liposomes, polymerized liposomes, other lipid vesicles, dendrimers, polyethylene glycol assemblies, polylysines, capped polylysines, poly(hydroxybutyric acid), dextrans, and coated polymers.

14. The targeted therapeutic agent of claim 1, wherein the linking carrier imparts a property to the agent selected from the group consisting of multivalency, enhanced circulation lifetimes, and increased payload.

15. The targeted therapeutic agent of claim 1, further comprising a stabilizing entity.

16. The targeted therapeutic agent of claim 15, wherein the stabilizing entity is dextran.

17. The targeted therapeutic agent of claim 1, wherein the therapeutic entity is selected from the group consisting of drugs, toxins, prodrugs, and radioactive isotopes.

18. The targeted therapeutic agent of claim 1, wherein the therapeutic entity is a radioactive isotope.

19. The targeted therapeutic agent of claim 18, wherein the radioactive isotope is selected from the group consisting of iodine-125, yttrium-90, yttrium-89, indium-111; technetium-99m, and europium-152.

20. The targeted therapeutic agent of claim 18, wherein the radioactive isotope is attached to the linking entity via a chelating group.

21. The targeted therapeutic agent of claim 20, wherein the chelating group is selected from the group consisting of DOTA, DTPA, ITC-DTPA, MX-DTPA, and citrate, and derivatives of DOTA, DTPA, ITC-DTPA, MX-DTPA, and citrate.

22. The targeted therapeutic agent of claim 1, wherein the therapeutic entity is selected from the group consisting of a chemotherapeutic agent, a toxin, and a prodrug.

23. A method of treating a disease accompanied by neovascularization, comprising the step of administering the targeted therapeutic agent of claim 1 to a subject in need of such administration.

24. The method of claim 23, wherein the step of administering the targeted therapeutic agent compromises the integrity of the vasculature associated with the pathology.

25. The method of claim 23, wherein the targeted therapeutic agent also carries a targeting entity against an additional target.

26. The method of claim 25, wherein the additional target is a cancer cell marker.

27. The method of claim 23, further comprising the step of administering an additional therapeutic agent simultaneously with or subsequent to the administering of the targeted therapeutic agent.

28. The method of claim 24, further comprising the step of administering an additional therapeutic agent simultaneously with or subsequent to the administering of the targeted therapeutic agent.

29. The targeted therapeutic agent of claim 1, wherein the linking carrier is capable of encapsulating additional materials.

30. The targeted therapeutic agent of claim 29, wherein the additional materials encapsulated in the linking carrier are selected from the group comprising nucleic acids, drugs, toxins, prodrugs, radioactive isotopes, and genes encoding proteins that exhibit cell toxicity.

31. The targeted therapeutic agent of claim 1, wherein the linking carrier is capable of attaching additional materials to the surface of the linking carrier.

32. The targeted therapeutic agent of claim 31, wherein the additional materials attached to the surface of the linking carrier are selected from the group comprising nucleic acids, drugs, toxins, prodrugs, radioactive isotopes, and genes encoding proteins that exhibit cell toxicity.
